(19) United States
(12) Patent Application Publication
(10) Pub. No.: US 2026/0335129 A1
(43) Pub. Date: Oct. 29, 2026

(54) HYPERTHERMIA TREATMENTS, DEVICES, PROCESSES, AND SYSTEMS

(71) Applicant: Starfish Neuroscience Inc., Bellevue, WA (US)

(72) Inventor: Jamu K. Alford, Port Orchard, WA (US)

(21) Appl. No.: 19/721,866

(22) Filed: Jun. 26, 2026

Related U.S. Application Data
(63) Continuation-in-part of application No. 19/288,807, filed on Aug. 1, 2025, now Pat. No. 12,708,556.
(60) Provisional application No. 63/679,392, filed on Aug. 5, 2024.

(51) Int. Cl. A61F 7/12; A61F 7/00

(52) U.S. Cl. CPC A61F 7/12; A61F 7/007; A61F 2007/0002; A61F 2007/0071; A61F 2007/0086; A61F 2007/0095; A61F 2007/126

(57) ABSTRACT

A hyperthermia treatment system includes an implantable medical device positioned in or adjacent to target tissue and a radiofrequency applicator outside a body. The implantable medical device includes a heating element, an implant coil arrangement, a temperature sensor, and wireless communication circuitry. The heating element includes a magnetic core, a resistive heating coil, or both and generates localized treatment-level heat from radiofrequency energy coupled through the implant coil arrangement. In some embodiments, a communication coil receives lower-power radiofrequency energy at a communication frequency to power the wireless communication circuitry and support temperature readout, while a separate heating coil receives higher-power radiofrequency energy at a different heating frequency and generates heat primarily through resistive losses. A control system receives temperature feedback and adjusts radiofrequency energy applied to the implantable medical device to regulate the localized treatment-level heat and maintain a desired temperature.

### CROSS REFERENCE TO RELATED APPLICATIONS

This application is a continuation-in-part of U.S. Patent Application No. 19/288,807, filed Aug. 1, 2025, which claims the benefit of priority to U.S. Provisional Patent Application No. 63/679,392, filed on Aug. 5, 2024. The entire contents of the aforementioned patent applications are herein incorporated by reference.

### TECHNICAL FIELD

The present disclosure relates to hyperthermia treatments, including implantable devices, external radiofrequency applicators, control systems, processes, medical treatments, and systems for applying controlled heat to target tissue in a body.

### BACKGROUND INFORMATION

Regional increases in body temperature for clinical use, sometimes referred to as hyperthermia therapy, can modify cellular behavior of body tissue by up-regulation or down-regulation of cellular functions and can, in some circumstances, destroy tissue by denaturing proteins and killing cells. Hyperthermia may be useful in oncology, for example, because some cancer cells are more sensitive to heat than healthy cells and because heat can increase sensitivity to chemotherapy, radiation therapy, immunotherapy, heat-triggered therapeutic agents, or other treatments.

In known systems, highly localized hyperthermia can be achieved inside the body by using focused ultrasound, and on the surface of the body by using heating pads with infrared lights. However, focused ultrasound systems can require complex phased-array ultrasound transmitters and may need to be used with magnetic resonance imaging thermometry. Such systems can be expensive, uncommon, and difficult to scale for treatment of large numbers of patients. Surface heating devices can achieve anatomical specificity at or near the skin, but are less effective for treating deeper anatomical structures. Attempting to use surface heating to reach deeper targets can result in off-target heating, surface overheating, or burns.

### SUMMARY OF THE DISCLOSURE

The present inventor has recognized that there remains a need for systems and devices that apply controlled hyperthermia to internal targets with improved localization, reduced complexity, wireless operation, temperature monitoring, and closed-loop or otherwise regulated temperature control.

Embodiments described herein include implantable medical devices, external radiofrequency applicators, control systems, and methods for applying controlled heat to target tissue in a body. As used herein, medical and therapeutic terminology encompasses both human medicine and veterinary medicine. A body comprises a human body or the body of a non-human animal. References to a patient encompass a human patient or a non-human animal receiving veterinary treatment. Implantable medical devices and the disclosed treatment systems, processes, and therapies are used to treat humans or non-human animals, including companion animals, livestock, research animals, and other mammals.

In some embodiments, an implantable medical device is positioned in or adjacent to tissue to be treated and generates localized heating using RF energy received from an external radiofrequency applicator. The implantable medical device is configured for extended presence in the body and repeated treatment sessions over days, weeks, or months. The implantable medical device is sized for injection or other minimally invasive placement and operates without an onboard battery in some embodiments. Battery-free wireless operation reduces implant size, avoids risks associated with battery chemicals, and avoids infection risk or routing complexity associated with a tether or electrical conductor extending through the body to an external power source.

In some embodiments, the implantable medical device includes an electrical circuit having an inductance-capacitance arrangement configured to harvest RF energy from electromagnetic waves transmitted by the external radiofrequency applicator. A magnetic core, such as a ferrite core, interacts with the inductance-capacitance arrangement to generate heat for treating tissue near the implantable medical device. Significant treatment-level heating occurs in the ferrite core in some embodiments, although additional heating occurs in a coil, capacitor, or other circuit element in some implementations.

As used herein, a magnetic core comprises a magnetic material configured to concentrate, guide, or otherwise interact with magnetic flux. The magnetic material comprises a ferromagnetic material, a ferrimagnetic material such as a ferrite material, or a combination thereof in different embodiments. A magnetic core associated with a heating coil is magnetically lossy and generates at least a portion of the localized treatment-level heat in some embodiments. A magnetic core associated with a communication coil improves RF-energy coupling, operating-power generation, communication performance, or combinations thereof.

As used herein, a heating element is a physical component or combination of physical components of an implantable medical device configured to convert energy derived from coupled RF energy into localized treatment-level heat. The heating element comprises a magnetically lossy material, an electrically resistive conductor, or both. Example heating elements include a magnetically lossy core, such as a ferrite core; a heating coil; a coil winding; a discrete resistor; a resistive conductor; a resistive wire; a resistive trace; a thin-film heater; a conductive polymer; or a combination thereof. The heating element generates heat through magnetic losses, resistive losses, or a combination of magnetic and resistive losses. In some embodiments, the heating element is separate from the implant coil arrangement. In other embodiments, at least a portion of the implant coil arrangement, such as a heating coil, also serves as the heating element.

In implant-side-regulated embodiments, the implantable medical device includes control circuitry, such as a microcontroller, and includes or communicates with a temperature sensor. The implantable medical device stores or receives information regarding a threshold temperature, maximum local temperature, desired treatment temperature, setpoint, treatment temperature range, thermal dose, or other treatment parameter. Based on sensed temperature or other treatment information, the implantable medical device selectively activates, deactivates, reduces, increases, or otherwise regulates heat generation.

In some embodiments, switch-controlled circuitry changes an energy-harvesting state of the implantable medical device. A semiconductor switch selectively activates, inserts, removes, bypasses, shunts, loads, or otherwise changes a circuit component that affects inductance, capacitance, resistance, loading, magnetic coupling, effective impedance, Q factor, resonant frequency, resonant response, or energy-harvesting efficiency. The change reduces treatment-level heating while allowing the implantable medical device to continue harvesting sufficient energy to operate the microcontroller or other circuitry. Some embodiments transition between a treatment-heating mode and a reduced-heating mode, while other embodiments include three or more power levels, heating states, or operating modes.

In some embodiments, the implantable medical device receives information using RF electromagnetic waves that also provide wireless power. Amplitude modulation of transmitted RF waves carries digital information to the implantable medical device in some implementations. The implantable medical device communicates information to the external radiofrequency applicator or a control system using backscatter communication, load modulation, RFID communication, or another wireless communication technique. Communicated information includes sensed temperature, implant identifier information, device status information, treatment information, or combinations thereof.

In externally regulated embodiments, the control system adjusts an RF signal applied to a radiofrequency coil based on temperature information received from the implantable medical device. The control system increases, decreases, maintains, or temporarily discontinues applied RF energy to maintain a desired device temperature, setpoint temperature, treatment temperature range, thermal dose, or other treatment condition. An implant coil arrangement includes a common implant coil that couples RF energy used both for heating and for supplying operating power to wireless communication circuitry in some embodiments. In other embodiments, the implant coil arrangement includes a heating coil and a separate communication coil operating at different frequencies. Implant-side regulation and external closed-loop power control are used together in some embodiments.

A plurality of implantable medical devices is positioned in or near one or more target regions in some embodiments. The implantable medical devices are operated simultaneously or at different times and independently regulate local heating based on sensed temperature, received RF energy, position relative to the external radiofrequency applicator, surrounding tissue, or another local condition. Different target regions thereby receive different heating profiles based on their respective sizes, locations, tissue characteristics, tumor types, treatment responses, or treatment requirements. In some embodiments, the external radiofrequency applicator transmits RF energy at a constant frequency, while each implantable medical device independently controls local heating. In other embodiments, the control system adjusts externally applied RF energy based on temperature information received from one or more implantable medical devices. External regulation and individual implant-side regulation thereby provide collective, regional, and local temperature control.

In externally regulated multiple-implant embodiments, different implantable medical devices are configured to receive treatment-level heating energy at different heating frequencies. For example, a first implantable medical device is tuned to a first heating frequency and a second implantable medical device is tuned to a second heating frequency. The control system independently adjusts RF power applied at the respective heating frequencies based on temperature information received from the corresponding implantable medical devices, thereby independently regulating heating without requiring an implant-side heat-reduction switch. The implantable medical devices use a common communication or temperature-readout frequency in some embodiments. The control system selectively obtains temperature information from individual implantable medical devices using implant identifiers, implant-specific commands, frequency-selective addressing, time-divided polling, or combinations thereof.

The disclosed devices, systems, and methods are useful in oncology and other therapeutic applications. In oncology, localized heating weakens or preconditions diseased cells, changes a tumor microenvironment, increases sensitivity to chemotherapy, radiation therapy, immunotherapy, heat-triggered therapeutic agents, or other treatments, activates temperature-sensitive therapeutic agents, or ablates tissue in some embodiments. Other applications include neurological applications, pain treatment, hypertension treatment, inflammation treatment, and other conditions in which controlled localized heating is useful.

Additional aspects and advantages will be apparent from the following detailed description of embodiments, which proceeds with reference to the accompanying drawings.

### BRIEF DESCRIPTION OF THE DRAWINGS

The accompanying drawings, which are incorporated herein and constitute part of this specification, are illustrative of particular embodiments of the present disclosure and do not limit the scope of the present disclosure. The drawings are not to scale and are intended for use in conjunction with the explanations in the following detailed description.

FIG. 1 is a block diagram showing a treatment system including an implantable medical device, an external radiofrequency applicator, a radiofrequency coil, and a control system, according to one embodiment.

FIG. 2 is a block diagram showing a plurality of implantable medical devices positioned in a body region to treat target tissue, according to one embodiment.

FIG. 3 is a schematic side view showing an implantable medical device including a magnetic core, a primary coil, a capacitor, a microcontroller, a switch, and a secondary coil, according to one embodiment.

FIG. 4 is an electrical circuit diagram showing electrical circuitry of an implantable medical device, including an energy-harvesting portion, a power-conditioning portion, and a secondary-coil switching arrangement, according to one embodiment.

FIG. 5 is a schematic side view showing an implantable medical device according to another embodiment, including a switch connected in parallel with a capacitor.

FIG. 6 is an electrical circuit diagram showing electrical circuitry of an implantable medical device, including capacitive or load-based circuitry for changing an energy-harvesting state, according to one embodiment.

FIG. 7 is an electrical circuit diagram showing electrical circuitry of an implantable medical device, including a Zener diode and semiconductor switch for load-based modification of an energy-harvesting state, according to one embodiment.

FIG. 8 is a pictorial view showing example relative sizes of implantable medical devices, according to one embodiment.

FIG. 9 is a pictorial treatment illustration showing a patient receiving infusion therapy while a radiofrequency coil wirelessly couples RF energy to an implantable medical device, and an example temporal relationship between hyperthermia treatment and chemotherapy, according to one embodiment.

FIG. 10 is a schematic side view of an implantable medical device having a temperature sensor, wireless communication circuitry, and an implant coil arrangement that supplies operating power to the wireless communication circuitry and couples RF energy for externally regulated treatment-level heating.

FIG. 11 is a schematic side view of an implantable medical device having separate heating and communication coils configured to operate at different frequencies for externally regulated temperature-feedback heating.

FIG. 12 is a schematic side view of an implantable medical device having separate heating and wireless temperature-communication portions associated with respective magnetic cores and separated by a gap.

FIG. 13 is a schematic side view of an implantable medical device having a heating portion and a wireless temperature-communication portion associated with respective magnetic cores and physically spaced apart within the implantable medical device.

FIG. 14 is a schematic side view of an implantable medical device having a resistive heating coil and a separate wireless temperature-communication portion configured to operate at different RF frequencies, according to one embodiment.

FIG. 15 is a flow chart of a process for controlling hyperthermia treatment of target tissue using an implantable medical device and an external radiofrequency applicator, according to one embodiment.

### DETAILED DESCRIPTION OF EMBODIMENTS

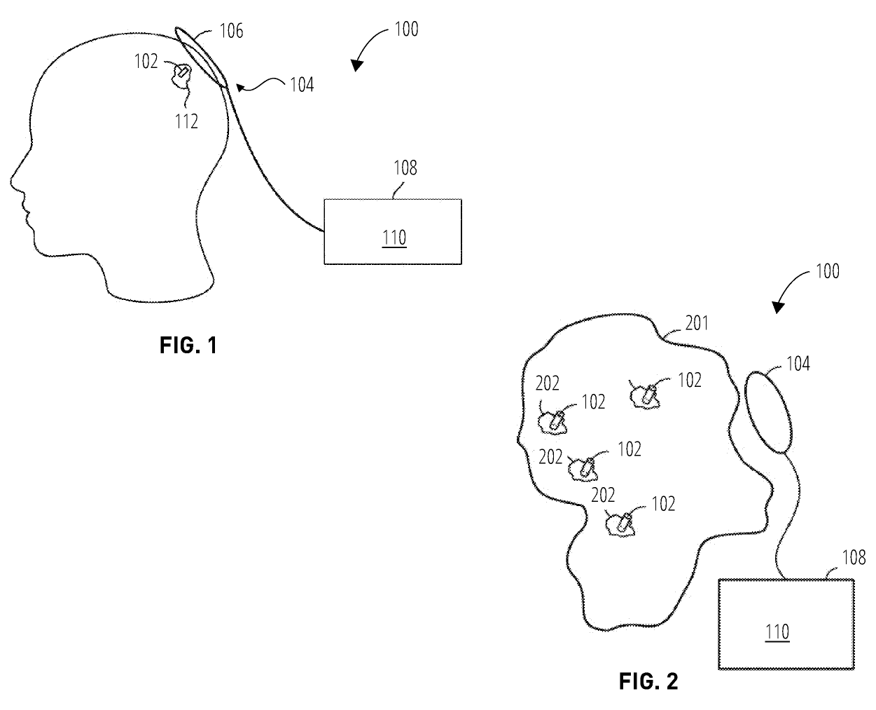

FIG. 1 shows treatment system 100 according to one embodiment. Treatment system 100 refers to the overall treatment arrangement and includes implantable medical device 102, external radiofrequency applicator 104 including radiofrequency coil 106, and control system 108 including control electronics 110.

Implantable medical device 102 refers to an implant, injectable device, implanted device, biomedical implant, or medical device configured to be positioned in a body. Implantable medical device 102 is also referred to herein as a medical treatment implant, heat-treatment implant, implant, injectable device, or biomedical implant. Implantable medical device 102 is injected or inserted into a body at specific locations and depths to create a local increase in temperature in or near tissue to be treated 112. In some embodiments, implantable medical device 102 operates to stay within a sensed temperature range of 37° C to 60° C, or within a narrower range such as about 38° C to 43° C for hyperthermia treatment. In some embodiments, implantable medical device 102 heats tissue to a higher temperature for ablation.

In the example treatment system 100, implantable medical device 102 facilitates at least four functions. First, implantable medical device 102 collects RF energy from an RF electromagnetic field generated using radiofrequency coil 106 and converts at least a portion of the collected RF energy to heat. Second, implantable medical device 102 senses a local temperature at or near implantable medical device 102. Third, implantable medical device 102 regulates or participates in regulating heat generation based on a maximum or desired treatment temperature, such as a setpoint specified by a clinician via control system 108 and stored on implantable medical device 102, stored by control system 108, or both. Fourth, implantable medical device 102 wirelessly communicates information, such as sensed temperature, device status, an implant identifier, or other treatment information, to control system 108.

In some embodiments, once implantable medical device 102 reaches a setpoint, implantable medical device 102 ceases or significantly reduces treatment-level heat generation until the sensed temperature falls below a threshold. In other embodiments, control system 108 uses temperature information from implantable medical device 102 to adjust an RF signal applied to radiofrequency coil 106. In still other embodiments, local implant-side control and external closed-loop control are used together to maintain a setpoint temperature or treatment temperature range.

Temperature information provides information regarding the state of implantable medical device 102. For example, a wireless readout indicating current temperature of implantable medical device 102 is used to confirm that treatment is being applied, verify actual device temperature, or determine whether implantable medical device 102 is receiving sufficient RF energy. If implantable medical device 102 fails to reach a setpoint temperature, the temperature information indicates that implantable medical device 102 is not receiving sufficient RF energy, such as due to incorrect placement of external radiofrequency applicator 104. Control system 108 uses this information to indicate to an operator that power should be increased, that an RF drive signal should be adjusted, that external radiofrequency applicator 104 should be positioned nearer to implantable medical device 102, or that another treatment parameter should be adjusted. In some embodiments, temperature information is also used to control the temperature of implantable medical device 102 without an internal setpoint, as described below.

Implantable medical device 102 is used singularly or in multiples around target tissue. For small targets, a single implantable medical device 102 is used. Implantable medical device 102 is injected, surgically implanted, or placed within a blood vessel. In some embodiments, implantable medical device 102 is configured like a stent or is placed to treat a nerve that wraps around a vessel, such as a renal nerve associated with hypertension.

For larger target regions, several implantable medical devices 102 are used to treat the entire volume. Multiple implantable medical devices 102 provide more uniform heating or heating of a larger volume of tissue. Because each implantable medical device 102 may be positioned at a different distance from radiofrequency coil 106 and therefore receive a different amount of RF energy, each implantable medical device 102 independently controls heating operation based on its local sensed temperature, or control system 108 regulates the RF signal applied to radiofrequency coil 106 based on temperature feedback from one or more implantable medical devices 102.

External radiofrequency applicator 104 refers to a device positioned external to the body and including or associated with radiofrequency coil 106. External radiofrequency applicator 104 includes RF electronics configured to apply an RF drive signal to radiofrequency coil 106, receive RF signals coupled through radiofrequency coil 106, or both. Radiofrequency coil 106 refers to a coil, antenna, transmit coil, receive coil, or transmit/receive coil configured to generate, receive, or couple RF electromagnetic signals. In some embodiments, external radiofrequency applicator 104 includes two coils, including a transmit coil and a separate receive coil. In other embodiments, a single coil provides both transmit and receive functions. In some embodiments, a non-resonant antenna external to the body is used to measure backscattered energy, load-modulated energy, or another signal from implantable medical device 102.

External radiofrequency applicator 104 is placed adjacent the body so that radiofrequency coil 106 wirelessly couples RF electromagnetic energy and communication signals with implantable medical device 102. Radiofrequency coil 106 generates an RF electromagnetic field from which implantable medical device 102 collects RF energy. Radiofrequency coil 106 also sends or receives RF signals to set temperature, receive temperature data, receive identifier data, or receive other data. The carrier frequency is selected to limit absorption of RF energy in tissue, such as absorption characterized by a specific absorption rate (SAR). The frequency is also chosen to comply with Federal Communications Commission regulations or similar regulations. In some embodiments, the RF carrier frequency is in a range of 100 kHz to 400 kHz. In other embodiments, the RF carrier frequency is in a broader range of 30 kHz to 14 MHz.

Control system 108 refers to control electronics 110, a computer, processor-based controller, user interface, RF control circuitry, or other electronics that control, monitor, or support treatment system 100. Control system 108 is coupled to external radiofrequency applicator 104 to control operation of external radiofrequency applicator 104, control an RF drive signal applied to radiofrequency coil 106, receive information communicated from implantable medical device 102 through external radiofrequency applicator 104, and verify actual device temperature. In some embodiments, control system 108 and external radiofrequency applicator 104 are separate components that communicate with one another. In other embodiments, control system 108 incorporates external radiofrequency applicator 104, or external radiofrequency applicator 104 incorporates at least a portion of control system 108.

Control system 108 includes, in some embodiments, an RF transmitter, RF receiver, transmitter/receiver pair, display, input device, keyboard, touchscreen, wired communication interface, wireless communication interface, Bluetooth interface, Wi-Fi interface, network interface, or Internet communication interface. Control system 108 also includes, in some embodiments, a processor and non-transitory computer-readable medium storing instructions that, when executed by the processor, cause control system 108 to perform one or more control, communication, temperature-monitoring, RF-drive, or treatment operations described herein.

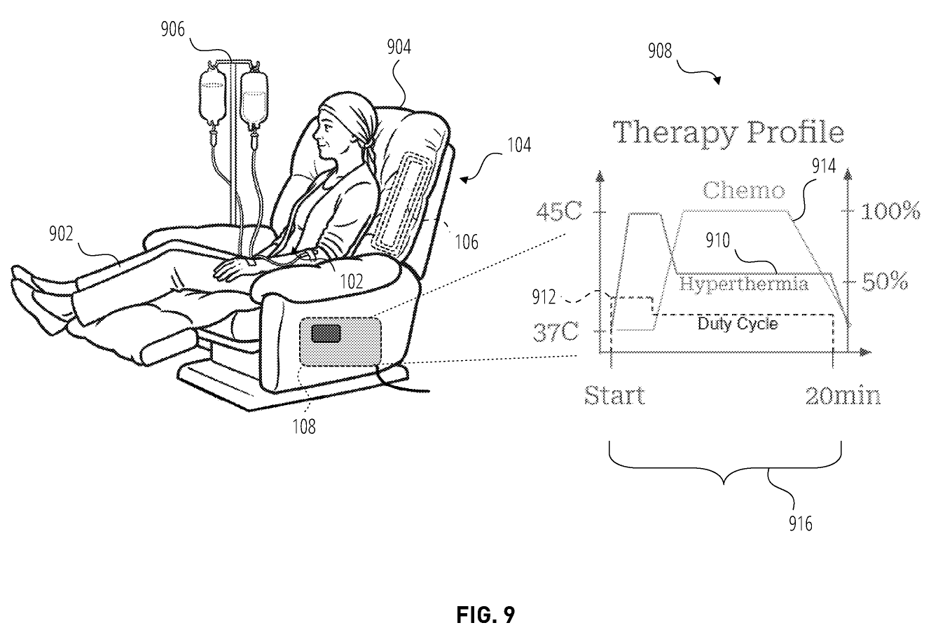

In some embodiments, control system 108 communicates treatment information through a communication network to a remote monitoring system (see, e.g., FIG. 9). The remote monitoring system comprises a remote computer, server, cloud-based computing system, clinician workstation, mobile device, caregiver device, or combinations thereof. The communicated treatment information comprises sensed temperature, temperature trends, implant identifier information, device status information, RF power information, treatment duration, treatment parameters, alerts, or combinations thereof. The remote monitoring system stores, displays, analyzes, or communicates the treatment information to a clinician, patient, caregiver, or other authorized user.

In some embodiments, control system 108 generates an alert when a sensed temperature, temperature trend, device status, communication condition, or treatment parameter satisfies an alert criterion. The alert is communicated through the network to the remote monitoring system or an authorized user. An authorized user provides treatment instructions, modifies a treatment parameter, schedules a subsequent treatment session, or requests additional temperature information through the remote monitoring system in some embodiments. Control system 108 applies locally stored safety limits or requires local authorization before implementing a remotely received treatment instruction.

FIG. 2 illustrates treatment system 100 in which multiple implantable medical devices 102 are positioned in a body region 201 to treat target tissue 202. Implantable medical devices 102 are positioned in spaced-apart locations and in locations sufficiently close to target tissue 202 to apply a desired level of heat. In some embodiments, implantable medical devices 102 are in direct physical contact with cancerous cells or other target tissue. In other embodiments, implantable medical devices 102 are positioned close enough to target tissue 202 to apply desired heat without direct physical contact.

Implantable medical devices 102 are positioned according to a predefined spatial pattern in some embodiments. The predefined spatial pattern comprises a linear pattern, planar pattern, three-dimensional array, circumferential pattern, grid, staggered pattern, pattern conforming to a boundary of target tissue 202, or another arrangement selected to provide a desired spatial distribution of heat. The positions, depths, orientations, and spacing of implantable medical devices 102 are determined before or during implantation based on a size, shape, location, or other characteristic of target tissue 202.

Implantable medical devices 102 are implanted manually, using image-guided instruments, using a robotic surgical system, or using a combination thereof. In some embodiments, a robotic surgical system receives implantation-plan information defining respective target locations, depths, orientations, or trajectories for implantable medical devices 102 and positions the implantable medical devices at the respective target locations. Imaging information obtained using ultrasound, fluoroscopy, computed tomography, magnetic resonance imaging, optical imaging, or another imaging modality is used to generate or update the implantation plan in some embodiments.

Adjacent implantable medical devices 102 are spaced so that heat generated by the adjacent implantable medical devices is conducted through intervening tissue and the resulting heated regions overlap. Two or more adjacent implantable medical devices 102 thereby cooperatively heat tissue positioned between or around the adjacent implantable medical devices. The spacing is selected based on heating-element output, treatment temperature, treatment duration, tissue thermal conductivity, tissue perfusion, implant orientation, implant depth, target geometry, or combinations thereof. In some embodiments, the predefined spatial pattern and spacing are selected using a thermal model or treatment-planning system to maintain a desired temperature or thermal dose throughout a selected volume of target tissue 202 while limiting heating of surrounding non-target tissue.

Each implantable medical device 102 has a respective identifier in some embodiments and communicates its identifier and sensed temperature to control system 108. Control system 108 receives the identifiers and temperature information to confirm that treatment is being performed and to associate received temperature information with the corresponding implantable medical device 102.

In some multiple-implant embodiments, radiofrequency coil 106 transmits RF energy at a common heating frequency corresponding to a tuned resonant frequency of each implantable medical device 102. Because the implantable medical devices 102 may receive different amounts of RF energy based on distance from radiofrequency coil 106, orientation, surrounding tissue, or other local conditions, each implantable medical device 102 independently activates, deactivates, reduces, or increases treatment-level heating based on its locally sensed temperature. In such embodiments, external radiofrequency applicator 104 transmits RF energy at a constant or otherwise commonly applied heating frequency, and individual implantable medical devices 102 regulate their respective temperatures locally.

In other multiple-implant embodiments, different implantable medical devices 102 are configured to receive treatment-level heating energy at different heating frequencies. For example, a first implantable medical device 102 is tuned to receive treatment-level heating energy at a first heating frequency, such as about 980 kHz, and a second implantable medical device 102 is tuned to receive treatment-level heating energy at a second heating frequency, such as about 990 kHz. Additional implantable medical devices 102 are tuned to additional heating frequencies in some embodiments. The stated frequencies and frequency separations are examples, and other frequencies and frequency separations are used.

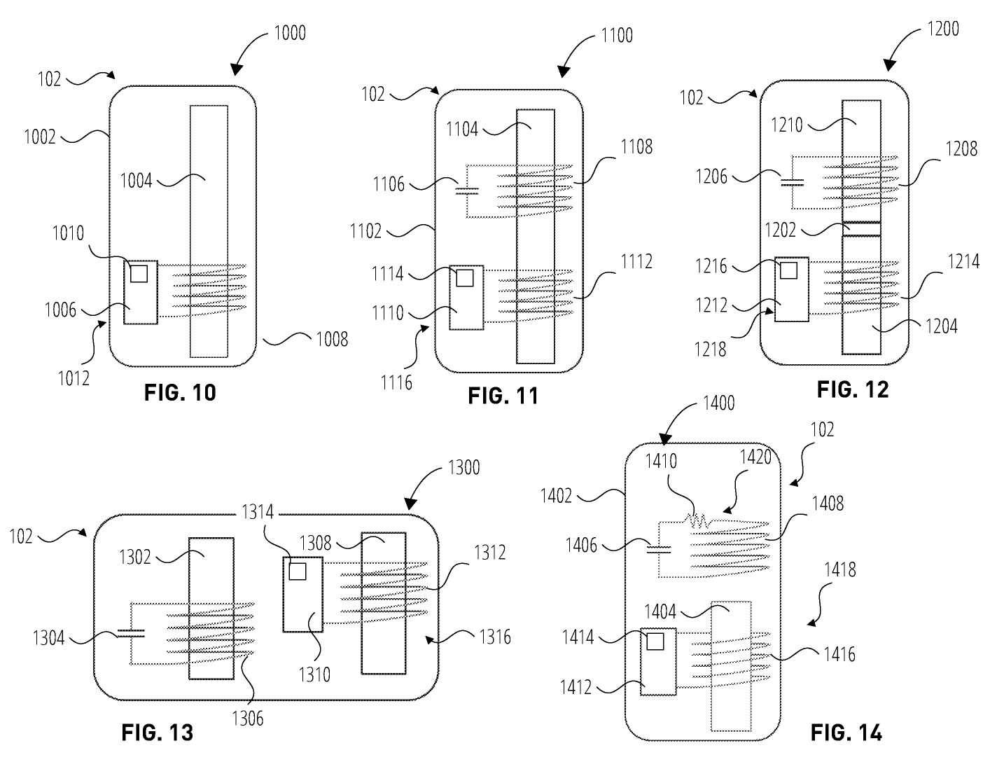

Control system 108 independently controls heating of the respective implantable medical devices 102 by adjusting a power level, duty cycle, application duration, pulse interval, or another operating parameter of RF energy applied at each heating frequency. RF energy at the different heating frequencies is applied sequentially, simultaneously, intermittently, or according to another coordinated sequence. Frequency-selective heating thereby permits the temperature of each implantable medical device 102 to be controlled individually through externally applied RF energy (see, e.g., FIGS. 10-14) without relying on implant-side regulation of the energy-harvesting state.

In some embodiments, the implantable medical devices 102 receive operating power for temperature sensing and wireless communication at a common communication or temperature-readout frequency, such as about 135 kHz. The common communication frequency is different from the respective heating frequencies in some implementations. Other communication frequencies are used in other embodiments.

To reduce or prevent overlapping transmissions from the implantable medical devices 102, control system 108 selectively obtains temperature information from individual implantable medical devices 102. In one implementation, control system 108 transmits an implant identifier, address, command, or other downlink information at the common communication frequency that instructs a selected implantable medical device 102 to communicate while the other implantable medical devices 102 remain silent. Control system 108 cycles through the implantable medical devices 102, records temperature information received from each selected implantable medical device 102, and updates the RF power applied at the heating frequency corresponding to each implantable medical device 102.

In another implementation, an implantable medical device 102 communicates temperature information when both RF energy at the common communication frequency and RF energy at the heating frequency assigned to that implantable medical device 102 are applied. The assigned heating frequency is applied at a reduced power level during temperature readout so that it identifies or enables the selected implantable medical device 102 without generating substantial treatment-level heat. For example, control system 108 applies the common communication frequency together with the first heating frequency to obtain temperature information from the first implantable medical device 102 and applies the common communication frequency together with the second heating frequency to obtain temperature information from the second implantable medical device 102.

A representative control cycle includes applying treatment-level RF energy at the first heating frequency, applying treatment-level RF energy at the second heating frequency, obtaining temperature information from the first implantable medical device 102, obtaining temperature information from the second implantable medical device 102, and independently updating the RF power applied at the first and second heating frequencies. These operations are repeated during treatment and occur in any order. For example, control system 108 initially applies substantially full power at each heating frequency, obtains temperature information from the respective implantable medical devices 102, and subsequently applies different reduced power levels at the respective heating frequencies based on the received temperature information.

In one example control cycle, control system 108 applies treatment-level RF energy at a first heating frequency, such as about 980 kHz, for an interval of about one second at an initial power level, and applies treatment-level RF energy at a second heating frequency, such as about 990 kHz, for an interval of about one second at an initial power level. Control system 108 then obtains temperature information from the respective implantable medical devices using an implant-specific downlink command, by applying a common temperature-readout frequency together with reduced-power RF energy at the heating frequency assigned to the selected implantable medical device, or both. Based on the respective temperature information, control system 108 adjusts the first and second heating-frequency power levels independently. For example, the control system subsequently applies RF energy at the first heating frequency at about 60% of the initial power level and RF energy at the second heating frequency at about 70% of the initial power level. The stated frequencies, durations, and power percentages are illustrative, and other frequencies, durations, power levels, and operating sequences are used.

In other embodiments, including single-implant embodiments or multiple-implant embodiments using regional external closed-loop control, control system 108 adjusts power delivered through external radiofrequency applicator 104 based on temperature feedback from one or more implantable medical devices 102. Control system 108 adjusts power, duty cycle, frequency, location, orientation, or another operating parameter of external radiofrequency applicator 104 based on the temperature feedback.

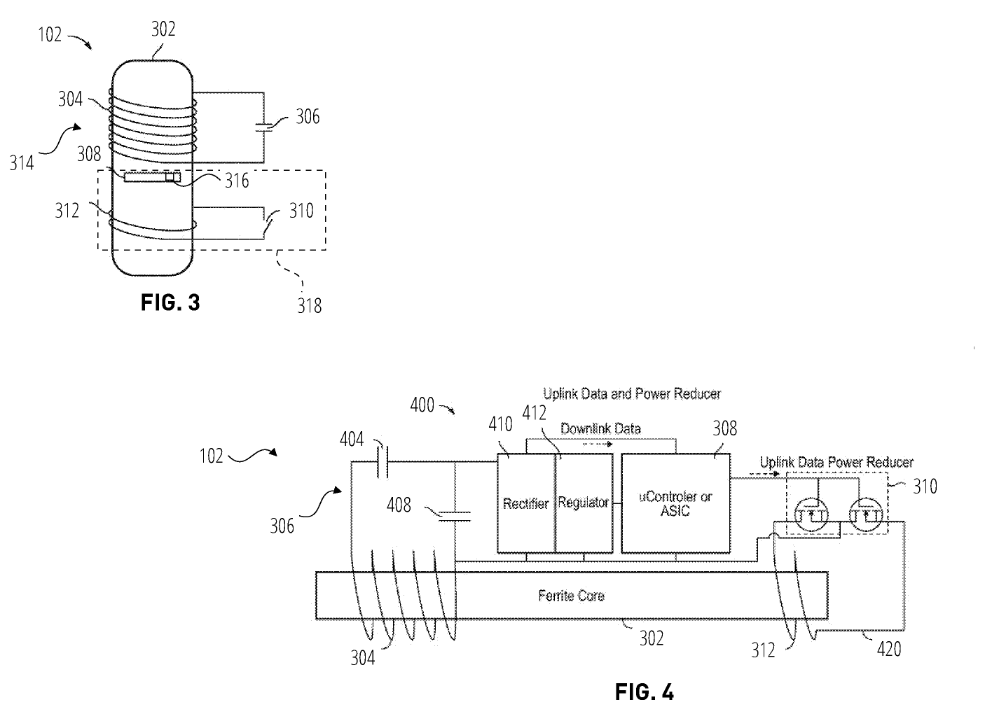

FIG. 3 shows implantable medical device 102 in greater detail, according to one embodiment. In this example, implantable medical device 102 includes magnetic core 302, primary coil 304, capacitor 306, microcontroller 308, switch 310, and secondary coil 312. Primary coil 304 and secondary coil 312 collectively provide an implant coil arrangement of implantable medical device 102. Primary coil 304 and capacitor 306 form an inductive-capacitive resonant circuit configured to couple and harvest RF energy from RF waves transmitted by external radiofrequency applicator 104 (FIG. 1). Secondary coil 312 is selectively activated to change magnetic coupling, resonant behavior, or energy harvesting of implantable medical device 102 and, in some embodiments, participates in wireless communication.

As used herein, an implant coil arrangement includes a single implant coil or a plurality of implant coils positioned within an implantable medical device and configured to perform one or more RF energy-coupling, operating-power, heating, communication, or energy-regulation functions. A single implant coil is used for more than one function in some embodiments. For example, a common implant coil couples RF energy used to generate treatment-level heat and supplies operating power to wireless communication circuitry. In other embodiments, the implant coil arrangement includes functionally distinct coils. Functionally distinct coils include, for example, a heating coil configured to couple RF energy used to generate treatment-level heat, a communication coil configured to couple RF energy used to supply operating power to wireless communication circuitry, and a secondary coil configured to change an energy-harvesting state or communicate information through load modulation or backscatter communication. A coil that supplies operating power to wireless communication circuitry additionally functions as an antenna or inductive-coupling element in some embodiments, while a separate antenna or coupling element is used in other embodiments.

The particular component or components that provide the heating element vary among the disclosed embodiments. In the embodiment of FIG. 3, heating element 314 comprises magnetic core 302 and, in some implementations, additionally comprises primary coil 304 or another electrically resistive conductor that generates a portion of the localized treatment-level heat. In other embodiments, including the embodiment of FIG. 14, the heating element comprises a heating coil, resistor, or other electrically resistive conductor and does not require a magnetic heating core. A component may perform more than one function. For example, a heating coil may form part of the implant coil arrangement by coupling RF energy and may also form part or all of the heating element by generating treatment-level heat through resistive losses.

Magnetic core 302 comprises ferrite or another magnetic material selected to concentrate magnetic flux and support resonant energy transfer. Magnetic core 302 is suitable for implantable medical device 102 because of its structural, magnetic, and mechanical properties.

In some embodiments, primary coil 304, capacitor 306, and magnetic core 302 form part of a resonant energy-harvesting structure having a selected quality factor, or Q factor. In some embodiments, the resonant energy-harvesting structure has a Q factor greater than or equal to 10. The Q factor represents energy stored in the resonant energy-harvesting structure relative to energy dissipated during oscillation. The selected Q factor allows implantable medical device 102 to efficiently couple RF energy, and loss mechanisms convert a portion of the coupled RF energy into heat. For example, magnetic losses in magnetic core 302 provide most treatment-level heating in some embodiments. In some embodiments, more than 80% of treatment-level heat is generated by magnetic core 302. Other electrical components may inherently generate heat during operation, but heat from those components is minimal relative to heat generated by magnetic core 302 in some embodiments. In some embodiments, additional heating occurs in primary coil 304, capacitor 306, or other circuit components.

In some embodiments, localized treatment-level heat is generated by a combination of magnetic losses in the magnetic core and resistive losses in one or more electrically conductive components of implantable medical device 102. The electrically conductive components include, for example, a coil winding, discrete resistor, resistive conductor, resistive trace, or another circuit element through which current derived from coupled RF energy flows. The relative contribution of the magnetic core and the electrically conductive components varies based on material composition, operating frequency, magnetic flux density, electrical resistance, current, geometry, and other operating parameters. In some embodiments, the magnetic core generates at least 50% of the localized treatment-level heat, and resistive losses in one or more other components generate a remaining portion of the localized treatment-level heat.

In other embodiments, the resistive heating element provides most or substantially all of the treatment-level heat, and the implantable medical device does not include a magnetic core. A resistive heating element includes a discrete resistor, resistive conductor, resistive wire, resistive trace, thin-film heater, conductive polymer, or another component having an electrical resistance selected to generate a desired amount of heat. Power-conditioning circuitry, switching circuitry, control circuitry, or combinations thereof regulate current, voltage, duty cycle, power, or another operating characteristic of the resistive heating element based on temperature information.

Implantable medical device 102 includes or is associated with a temperature sensor 316 that determines a current temperature at, in, or near implantable medical device 102. Temperature sensor 316 is integrated into microcontroller 308, included on the same substrate as microcontroller 308, electrically coupled to microcontroller 308, or physically separate from microcontroller 308. In some embodiments, sensed internal device temperature is correlated with or converted to an expected biological temperature external to implantable medical device 102. Accordingly, sensed temperature represents actual local tissue temperature, heating-element temperature, device temperature, or a value correlated with any of these temperatures.

Microcontroller 308, or other control circuitry of implantable medical device 102, measures or receives local device temperature, receives and stores a maximum temperature setpoint via a wireless signal, controls the state of switch 310 to report temperature, and controls the state of switch 310 to stop or reduce heating when a maximum temperature is reached. Some implementations use only a subset of these features.

Microcontroller 308 is implemented, in some embodiments, as a semiconductor integrated circuit, application-specific integrated circuit (ASIC), field-programmable gate array (FPGA), microprocessor, microcontroller, logic circuit, or combination thereof. Microcontroller 308 includes memory in some embodiments. The memory stores temperature setpoints, identifiers, treatment parameters, instructions, calibration values, or other information used by implantable medical device 102. In some embodiments, memory, rectifier circuitry, regulator circuitry, switch-control circuitry, communication circuitry, temperature-sensing circuitry, or combinations thereof are fixed on a common support, integrated with microcontroller 308, incorporated into a single ASIC, or provided by two or more discrete components. Components such as a coil, magnetic core, capacitor, or Zener diode are separate from integrated-circuit components in some embodiments.

Information regarding a maximum local temperature, threshold temperature, treatment temperature range, setpoint, or thermal dose is stored on implantable medical device 102, such as in memory associated with microcontroller 308. Implantable medical device 102 uses the stored information to selectively turn on, turn off, reduce, or increase heat treatment generated by magnetic core 302 or another heating element.

Switch 310 is implemented using a semiconductor switch, a mechanical switch, or another switching arrangement configured to selectively open or close a current path. In some embodiments, switch 310 is implemented using at least one low on-resistance transistor, such as a MOSFET, or a solid-state electronic relay. A semiconductor implementation reduces moving parts in implantable medical device 102 and reduces size, complexity, or reliability concerns associated with mechanical switching arrangements. As used herein, a semiconductor switch means a switch made using a semiconductor fabrication process and without incorporating a mechanical arm or bar to change the current path.

Switch 310 and secondary coil 312 change the coupling between implantable medical device 102 and the RF electromagnetic field. Secondary coil 312, which is also referred to as a choke coil, is magnetically coupled to magnetic core 302 and primary coil 304. Secondary coil 312 has fewer turns than primary coil 304 in some embodiments to reduce voltage seen by switch 310. When switch 310 activates secondary coil 312, current induced in secondary coil 312 produces a magnetic field that opposes or modifies magnetic flux associated with primary coil 304. Activation of secondary coil 312 thereby changes the effective impedance, resonant behavior, magnetic coupling, or energy-harvesting efficiency of implantable medical device 102. As a result, implantable medical device 102 harvests less RF energy for treatment-level heating, while continuing to harvest sufficient energy to operate microcontroller 308 or other control circuitry.

During a heating cycle, switch 310 is in a state that permits primary coil 304 and capacitor 306 to harvest RF energy efficiently enough to generate treatment-level heat. In some embodiments, switch 310 is in an open condition most of the time during the heating cycle and is closed briefly to send temperature information to control system 108. During a cooling or reduced-heating cycle, when implantable medical device 102 has reached a setpoint temperature, switch 310 activates secondary coil 312 to reduce treatment-level heating. In some embodiments, switch 310 is in a closed condition most of the time during the cooling or reduced-heating cycle and opens briefly to send temperature information to control system 108. An open, off, or deactivated state of a semiconductor switch does not require zero current and includes a state having negligible leakage current.

In the reduced-heating state, implantable medical device 102 is not necessarily completely decoupled from the RF electromagnetic field. Rather, conversion of RF energy to treatment-level heat is reduced, detuned, damped, opposed, or otherwise limited so that temperature decreases or is maintained below a threshold. In some embodiments, more than 90% of RF energy that would otherwise be converted to treatment-level heating is rejected or reduced in an off state, although residual heating occurs in some implementations. For example, when secondary coil 312 is activated, an electromagnetic field generated by secondary coil 312 interacts with a field of primary coil 304 and modifies the field of primary coil 304. The field of primary coil 304 is shifted or pushed out of an optimal energy-harvesting position. Power produced from wireless-energy conversion is then reduced to a level sufficient to operate microcontroller 308, but insufficient to produce treatment-level heat.

The heating response of implantable medical device 102 is governed at least in part by analog electrical and thermal behavior. A transition between a heating state and a reduced-heating state need not be instantaneous and may involve a transition period as energy dissipates in magnetic core 302 or another component. In an off state or reduced-heating state, implantable medical device 102 continues harvesting some energy from transmitted RF waves to supply operation of microcontroller 308. The harvested energy interacts with magnetic core 302 at a lower level insufficient to produce treatment-level heating.

In some embodiments, the external RF wave is transmitted at a constant frequency that corresponds to the resonant frequency of primary coil 304 and capacitor 306 when implantable medical device 102 is in a heating mode. Transmitting at a constant frequency supports operation of multiple implantable medical devices 102 in a treatment region.

Switch 310 also provides, in some embodiments, a communication function. Low-frequency switching, such as switching on the timescale of seconds based on measured temperature, regulates heating. Higher-frequency switching, such as switching on the timescale of milliseconds, modulates the electromagnetic field coupled to external radiofrequency applicator 104 and communicates data to control system 108 without substantially changing the thermal state of implantable medical device 102. Example switching frequencies for signaling are 100 Hz to 100 kHz. Because the temperature of magnetic core 302 changes more slowly than the signaling rate, the higher-frequency switching communicates information while having little effect on average heating. In some embodiments, rapid opening and closing of switch 310 sends digital information from implantable medical device 102 to external radiofrequency applicator 104 through fluctuations in an RF magnetic field. This communication technique is inductive backscatter or another form of backscatter communication.

In some embodiments, wireless communication circuitry 318 includes microcontroller 308, switch 310, secondary coil 312, other circuitry configured to vary load, power consumption, resonant response, magnetic coupling, or electromagnetic-field coupling of implantable medical device 102 to communicate sensed temperature, an implant identifier, device status, or other information to external radiofrequency applicator 104 or control system 108.

Implantable medical device 102 receives information using electromagnetic waves that also provide wireless power. For example, amplitude modulation of transmitted RF waves carries digital information to implantable medical device 102. Implantable medical device 102 includes a demodulation process using a rectifier, regulator, and microcontroller to identify digital data bits in the transmitted waves. The digital data includes a temperature setpoint, a maximum temperature, an identifier, timing information, treatment parameters, or other control information.

Implantable medical device 102 transmits information using backscatter communication. For example, implantable medical device 102 selectively varies operation of electronics onboard implantable medical device 102, and the resulting variation changes an electromagnetic field monitored by external radiofrequency applicator 104 or control system 108. Control system 108 demodulates information from implant-initiated variations in the field. In some embodiments, implantable medical device 102 varies current load or power consumption among two or more levels to communicate digital information such as a sensed temperature or implant identifier.

In some embodiments, particularly where a single implantable medical device 102 is used and provides real-time temperature feedback to control system 108, control system 108 adjusts power delivered through external radiofrequency applicator 104 based on temperature feedback from implantable medical device 102 to maintain a desired temperature. In such an embodiment, the sensed temperature reflects the temperature of a single heating element of implantable medical device 102. Control system 108 implements closed-loop control using real-time or near-real-time communication from implantable medical device 102, including constant, continuous, or periodic temperature updates sufficient to maintain the desired temperature without variation that would affect treatment performance. In some implementations (see, e.g., FIGS. 10-14), implant-side heat-reduction circuitry, such as switch 310, secondary coil 312, or another choke coil or switch-controlled heat-reduction circuit, is omitted because temperature is controlled through external power regulation.

FIG. 4 illustrates electrical circuitry 400 of implantable medical device 102 according to one embodiment. Electrical circuitry 400 harvests energy from RF waves received from external radiofrequency applicator 104, generates treatment-level heat using magnetic core 302, powers microcontroller 308, and selectively reduces heat generation based on sensed temperature.

Electrical circuitry 400 includes an energy-harvesting portion formed at least in part by primary coil 304, first capacitor 404, and second capacitor 408. Primary coil 304 is an inductor and, together with first capacitor 404 and second capacitor 408, establishes a desired resonant frequency for electrical circuitry 400. External radiofrequency applicator 104 transmits RF waves at or near the desired resonant frequency. As used herein, RF waves transmitted at or near a resonant frequency include RF waves transmitted at the resonant frequency and RF waves transmitted within a tolerance of the resonant frequency. In some embodiments, the tolerance is about ±10% of the resonant frequency.

Electrical circuitry 400 receives the RF waves and generates RF current that causes magnetic core 302 to generate heat at a desired level to treat tissue touching, positioned adjacent to, or otherwise sufficiently near implantable medical device 102.

Electrical circuitry 400 also includes a power-conditioning portion that supplies operating power to microcontroller 308. Second capacitor 408 operates as a voltage divider to prevent damage to rectifier 410 in some embodiments. A portion of current generated by primary coil 304 and associated capacitors flows to rectifier 410. Rectifier 410 rectifies an AC signal generated from received RF waves, and regulator 412 conditions the rectified signal to provide a stable DC voltage power supply to microcontroller 308. Circuitry in rectifier 410 or elsewhere in electrical circuitry 400 filters digital signals modulated into the transmitted RF waves to recover digital ones and zeros. Such signals carry information, such as temperature thresholds, setpoints, identifiers, timing information, treatment parameters, or other control values, to microcontroller 308 for storage or use by microcontroller 308.

Microcontroller 308 includes a temperature sensor or receives temperature information from another sensor. Microcontroller 308 controls switch 310 based on the temperature information. In some embodiments, switch 310 includes a pair of MOSFETs arranged as a bidirectional switch for an RF current path associated with secondary coil 312 and line 420. Because current induced in secondary coil 312 by the RF electromagnetic field is an alternating current having positive and negative directions, the pair of MOSFETs is arranged to control current flow in both directions.

In the illustrated embodiment, a common node between the pair of MOSFETs is electrically coupled to a conductor associated with primary coil 304, and drain-side nodes of the pair of MOSFETs are electrically coupled to portions of a secondary-coil circuit including secondary coil 312 and line 420. Line 420 forms part of a selectively activatable current path associated with secondary coil 312. When switch 310 is activated, secondary coil 312 is electrically loaded through switch 310.

Secondary coil 312 is wrapped around magnetic core 302 and magnetically coupled with primary coil 304. When switch 310 is open, secondary coil 312 is electrically inactive or lightly loaded, and primary coil 304, first capacitor 404, and second capacitor 408 operate as a resonant energy-harvesting circuit at or near the frequency of RF waves transmitted by external radiofrequency applicator 104. When switch 310 is closed, secondary coil 312 is electrically inserted, shunted, shorted, loaded, or otherwise activated through switch 310 and line 420. Depending on the winding direction of secondary coil 312, the polarity of connections to secondary coil 312, and the circuit node through which secondary coil 312 is connected, activation of secondary coil 312 changes an effective inductance, effective impedance, damping, Q factor, resonant frequency, or resonant response of electrical circuitry 400. As a result, electrical circuitry 400 is less efficiently resonant at the frequency of the RF waves transmitted by external radiofrequency applicator 104, harvests less RF energy for treatment-level heating, and reduces heat generated by magnetic core 302.

Activation or insertion of secondary coil 312 reduces energy harvesting to a second reduced level because the resonant behavior of electrical circuitry 400 changes while the frequency of external RF waves remains unchanged. In some embodiments, the reduction is a significant reduction, such as about 90%. The reduced level is sufficient to supply power to microcontroller 308 through rectifier 410 and regulator 412, but insufficient to generate treatment-level heat with magnetic core 302. In a low-power or treatment-off mode, sensed temperature is allowed to drop to a threshold or range. When microcontroller 308 determines that the threshold has been reached, microcontroller 308 signals switch 310 to open and disable secondary coil 312, thereby reactivating heat treatment.

Switch 310 also transmits information to external radiofrequency applicator 104 or control system 108 using backscatter communication in some embodiments. Switch 310 switches at high frequency to modify the electromagnetic field of electrical circuitry 400 and communicate digital information. External radiofrequency applicator 104 or control system 108 monitors the electromagnetic field and detects information including an implant identifier, sensed temperature, device status, or other data.

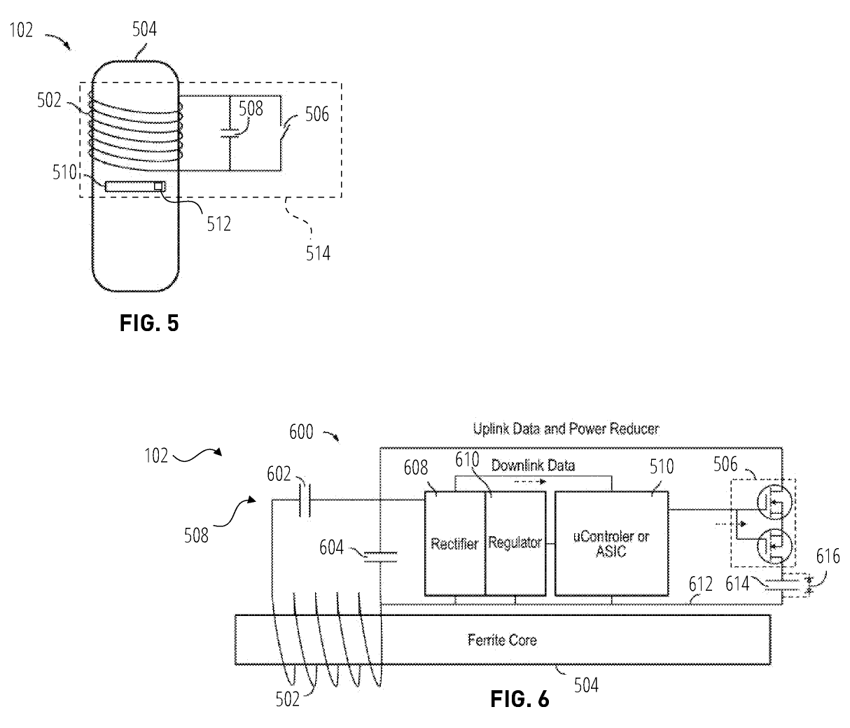

FIG. 5 illustrates implantable medical device 102 according to another embodiment. The embodiment of FIG. 5 is similar to the embodiment of FIG. 3, except there is a single coil 502 around a magnetic core 504 and a switch 506 is connected in parallel with a capacitor 508. When switch 506 is open, capacitor 508 forms part of a resonant energy-harvesting circuit of implantable medical device 102. When switch 506 is closed, switch 506 bypasses capacitor 508 and changes the resonant behavior of implantable medical device 102. In this state, implantable medical device 102 is detuned from, damped at, or otherwise less efficiently coupled to RF energy transmitted by external radiofrequency applicator 104. As a result, implantable medical device 102 reduces energy harvested for treatment-level heating.

Because switch 506 is connected in parallel with capacitor 508, switch 506 is exposed to voltage present across capacitor 508 when switch 506 is open. Accordingly, in some embodiments, switch 506 is implemented as a high-voltage semiconductor switch having a voltage rating selected to withstand the voltage present across capacitor 508.

Microcontroller 510 controls switch 506 to change operation of implantable medical device 102 between a treatment-heating mode and a reduced-heating or hold mode. In the treatment-heating mode, switch 506 is open and implantable medical device 102 harvests RF energy at a level sufficient to generate treatment-level heat using a magnetic core. In the reduced-heating or hold mode, switch 506 is closed and capacitor 508 is bypassed, thereby reducing, limiting, or turning off treatment-level heating for treating cells. In some embodiments, implantable medical device 102 continues receiving sufficient energy to operate microcontroller 510 while treatment-level heating is reduced or off. When a temperature sensor 512 detects that the temperature drops to a threshold or range, microcontroller 510 changes the state of switch 506 to return implantable medical device 102 to the treatment-heating mode.

In some embodiments, implantable medical device 102 includes wireless communication circuitry 514. Wireless communication circuitry 514 includes microcontroller 510, switch 506, capacitor 508, coil 502, associated conductors, or combinations thereof. Wireless communication circuitry 514 communicates sensed temperature, an implant identifier, device status, or other information to external radiofrequency applicator 104 or control system 108 by varying load, power consumption, resonant response, impedance, magnetic coupling, or electromagnetic-field coupling of implantable medical device 102.

FIG. 6 illustrates electrical circuitry 600 of implantable medical device 102 according to another embodiment. Electrical circuitry 600 harvests RF energy from external radiofrequency applicator 104, generates treatment-level heat using magnetic core 504, powers microcontroller 510, and selectively changes an energy-harvesting state of implantable medical device 102 using capacitive or load-based modification.

Electrical circuitry 600 includes an energy-harvesting portion formed at least in part by coil 502, first capacitor 602, second capacitor 604, and magnetic core 504. Coil 502, together with first capacitor 602 and second capacitor 604, establishes a desired resonant frequency for electrical circuitry 600. External radiofrequency applicator 104 transmits RF waves at or near the desired resonant frequency. The RF waves induce an RF voltage and RF current in coil 502 and the associated resonant network. The induced RF current produces magnetic flux in magnetic core 504 and causes magnetic core 504 to generate heat at a desired or determined level. In some embodiments, the generated heat treats tissue touching, positioned adjacent to, or otherwise sufficiently near implantable medical device 102. Example treatment conditions include a temperature increase of up to about 20° C above baseline, collected power of up to about 5 W, or a two-hour treatment involving about 36,000 J.

Electrical circuitry 600 also includes a power-conditioning portion that supplies operating power to microcontroller 510. In some embodiments, first capacitor 602 and second capacitor 604 form a capacitive voltage divider or resonant capacitive network, and a tap or node of the capacitive network provides an RF signal to rectifier 608. Rectifier 608 rectifies the RF signal to generate a rectified signal, and regulator 610 conditions the rectified signal to provide a stable DC voltage to microcontroller 510. Circuitry in rectifier 608 or elsewhere in electrical circuitry 600 rectifies, filters, demodulates, or otherwise processes digital signals modulated into the transmitted RF waves to recover digital ones and zeros. Amplitude modulation of RF waves transmitted by external radiofrequency applicator 104 carries digital data to electrical circuitry 600. The digital data includes temperature values, thresholds, setpoints, timing values, identifiers, treatment parameters, or other control information for storage or use by microcontroller 510.

Microcontroller 510 includes a temperature sensor or receives temperature information from another temperature sensor associated with electrical circuitry 600. The temperature sensor determines a current temperature at, in, or near implantable medical device 102. In some embodiments, microcontroller 510 correlates or converts a sensed internal temperature to an expected biological temperature external to implantable medical device 102. Accordingly, sensed temperature represents a current implant temperature, heating temperature, local tissue temperature, or value correlated with one or more of these temperatures. Microcontroller 510 uses the sensed temperature to determine when treatment-level heating is turned on, turned off, reduced, or resumed.

Microcontroller 510 controls semiconductor switch 506 to change an energy-harvesting state of electrical circuitry 600. In some embodiments, semiconductor switch 506 includes a pair of MOSFETs, and microcontroller 510 outputs a control signal that turns both MOSFETs on or off together. When semiconductor switch 506 is closed, line 612 establishes a switched current path associated with second capacitor 604. In some embodiments, the switched current path is parallel to second capacitor 604. Closing semiconductor switch 506 reduces or eliminates current flow through second capacitor 604, bypasses at least a portion of second capacitor 604, changes current flow through the capacitive network, changes the effective capacitance of electrical circuitry 600, or otherwise changes the resonant behavior of electrical circuitry 600.

Additional capacitor 614 is inserted, activated, or coupled into electrical circuitry 600 when semiconductor switch 506 is closed in some embodiments. Activation of additional capacitor 614 changes the effective capacitance of electrical circuitry 600 and modifies the resonant frequency established by coil 502 and the capacitors of electrical circuitry 600, including first capacitor 602, second capacitor 604, and additional capacitor 614. In some embodiments, a resonant-frequency relationship based on inductance and capacitance is used to calculate or select the change in resonant frequency caused by activation of additional capacitor 614. External radiofrequency applicator 104 continues transmitting RF waves at the original resonant frequency, while electrical circuitry 600 shifts to a different resonant frequency or otherwise becomes less efficiently resonant at the transmitted frequency. This frequency misalignment reduces current flowing through coil 502, reduces energy harvested by electrical circuitry 600, and transitions implantable medical device 102 to a lower-power state sufficient to power microcontroller 510 while reducing, limiting, or turning off treatment-level heating. This operation is an example of capacitive modulation or capacitive modification used to disrupt energy harvesting.

The change in harvested energy between modes reduces energy generation by a significant amount in some embodiments, such as about 90%, thereby reducing or turning off heat treatment generated by magnetic core 504. In a low-power or treatment-off mode, sensed temperature drops to a threshold or range. When microcontroller 510 determines that the threshold has been reached, microcontroller 510 signals semiconductor switch 506 to open and disables the current path through line 612, thereby returning electrical circuitry 600 to a treatment-heating mode.

One or more Zener diodes 616 are included in electrical circuitry 600 in some embodiments. In some embodiments, back-to-back Zener diodes 616 replace additional capacitor 614. In other embodiments, electrical circuitry 600 includes both additional capacitor 614 and back-to-back Zener diodes 616, such as in series. Back-to-back Zener diodes 616 provide a bidirectional voltage-limiting load in the switched current path associated with line 612. The back-to-back arrangement limits voltage in both polarities of an RF waveform and causes the switched current path to conduct differently at different voltage levels. As a result, the switched current path changes the loading or resonant behavior of electrical circuitry 600 without operating as an uncontrolled hard short across second capacitor 604 or the power-conditioning portion of electrical circuitry 600. This arrangement maintains sufficient voltage to operate microcontroller 510 while changing the energy-generation state of electrical circuitry 600. The Zener-diode arrangement provides load-based modulation by inserting or activating a voltage-limiting load that changes the amount of RF energy harvested by electrical circuitry 600.

Electrical circuitry 600 operates in a manner similar to electrical circuitry 400 (FIG. 4) with respect to sending and receiving digital signals and related data. Electrical circuitry 600 also operates in a manner similar to electrical circuitry 400 with respect to the rectifier, regulator, and microcontroller arrangement used to derive operating power from RF waves received by implantable medical device 102.

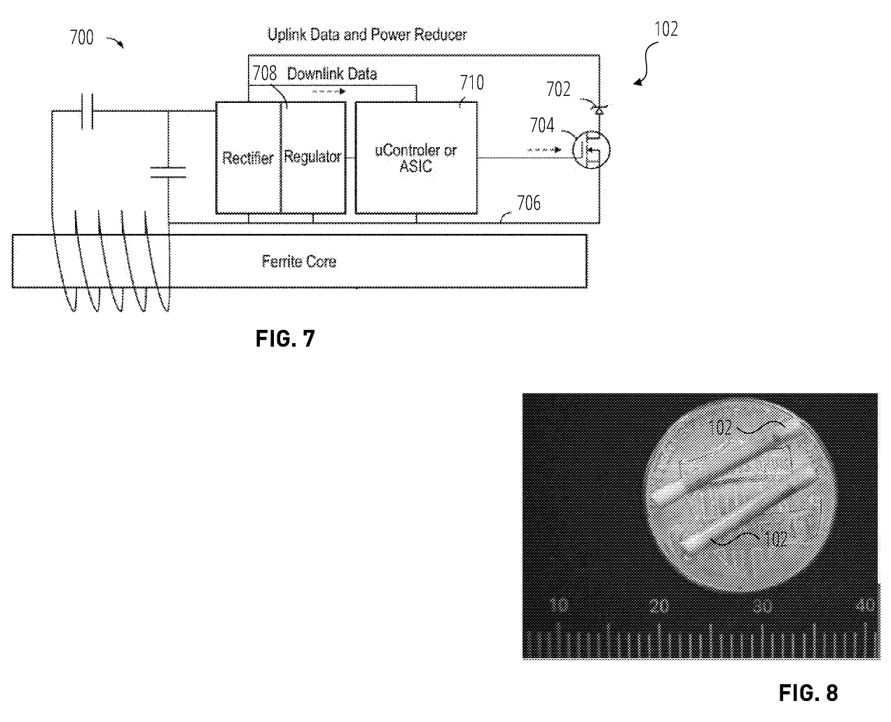

FIG. 7 illustrates electrical circuitry 700 of implantable medical device 102 according to another embodiment. Electrical circuitry 700 is similar to electrical circuitry 600 (FIG. 6), except that electrical circuitry 700 uses Zener diode 702 and semiconductor switch 704 to provide load-based modification of an energy-harvesting state. In some embodiments, semiconductor switch 704 is implemented as a MOSFET.

Zener diode 702 operates as a voltage-regulating or voltage-limiting element in a switched current path associated with line 706. When semiconductor switch 704 is closed, Zener diode 702 is coupled into the switched current path and changes loading of electrical circuitry 700. The added loading disrupts or reduces the ability of electrical circuitry 700 to harvest energy from RF waves transmitted by external radiofrequency applicator 104. At the same time, Zener diode 702 limits voltage in the switched current path so that regulator 708 continues receiving sufficient voltage, current, or power to operate microcontroller 710. This operation transitions electrical circuitry 700 to a reduced-heating or hold mode in which treatment-level heating is reduced or turned off while microcontroller 710 remains powered.

The Zener-diode arrangement provides load modulation or load modification to disrupt energy harvesting. In some embodiments, semiconductor switch 704 selectively inserts, activates, or couples Zener diode 702 into electrical circuitry 700 to change an energy-generation state of implantable medical device 102. When semiconductor switch 704 is opened, Zener diode 702 is removed from, deactivated in, or less strongly coupled to the switched current path, and electrical circuitry 700 returns to a treatment-heating mode.

Any of the inductive, capacitive, or load-modulation embodiments described herein may be combined. Inductive modulation, capacitive modulation, and load modulation refer to the primary modulation mechanism in a given embodiment and do not exclude additional modulation or changes to inductance, capacitance, resistance, loading, magnetic coupling, impedance, Q factor, resonant frequency, resonant response, or other operating characteristics.

FIG. 8 illustrates example relative sizes of implantable medical device 102. In some embodiments, implantable medical device 102 has a diameter of about 1.5 mm and a length of about 14 mm. In other embodiments, implantable medical device 102 has a length in a range of about 5 mm to about 20 mm and a diameter in a range of about 1 mm to about 3 mm. The size of implantable medical device 102 is selected based on target tissue, implantation technique, required power, communication range, heating requirements, available implant volume, patient anatomy, or other design considerations.

Implantable medical device 102 includes an enclosure, casing, housing, capsule, or other sealed structure that encloses electronics and prevents liquid from reaching liquid-sensitive components. The sealed structure includes one or more materials that permit RF waves to reach internal electrical circuitry without blocking or substantially interfering with the RF waves. Example materials include glass, alumina, ceramic, polymer, biocompatible materials, and combinations thereof. Epoxy or another sealing material seals the structure in some embodiments.

In some embodiments, implantable medical device 102 has a cylindrical shape suitable for injection into the body. Other shapes are used in other embodiments depending on the target tissue, implantation route, desired orientation, fixation requirements, or manufacturing considerations. The material of the sealed structure is biocompatible or otherwise suitable for long-term presence in the body. In some embodiments, features are added to an outside surface of implantable medical device 102 to prevent migration in the body, promote tissue engagement, facilitate removal, or combinations thereof.

FIG. 9 illustrates an example treatment environment and therapy profile for implantable medical device 102. In the illustrated environment, a patient 902 receives an infusion treatment while seated in treatment chair 904. Infusion system 906 delivers a therapeutic agent, such as a chemotherapy agent, to the patient. Radiofrequency coil 106 is incorporated into, positioned on, or positioned adjacent to treatment chair 904 and is coupled to external radiofrequency applicator 104 and control system 108. Radiofrequency coil 106 wirelessly couples RF energy to one or more implantable medical devices 102 positioned in the patient.

Therapy profile 908 graphically illustrates an example temporal relationship between hyperthermia treatment and chemotherapy. Hyperthermia temperature profile 910 represents temperature at or near implantable medical device 102, temperature of target tissue, or another temperature correlated with treatment. In the illustrated example, temperature begins near normal body temperature, increases to a treatment temperature in response to a duty cycle 912, and is thereafter reduced or maintained during at least a portion of the treatment interval at a lower duty cycle 912. The illustrated temperatures and timing are examples and are not limiting. A hyperthermia treatment may use a lower maximum temperature, a higher maximum temperature, a substantially constant treatment temperature, repeated heating and cooling cycles, or another temperature profile selected for a particular therapy.

Chemotherapy profile 914 schematically represents timing of administration or presence of a chemotherapy agent relative to the hyperthermia treatment. Chemotherapy profile 914 is not necessarily plotted on the temperature scale and instead indicates that chemotherapy and hyperthermia overlap during at least part of treatment interval 916. Hyperthermia treatment is applied before, during, after, or otherwise in temporal association with administration of chemotherapy.

During a treatment session, external radiofrequency applicator 104, under control of control system 108, provides RF energy through radiofrequency coil 106 to implantable medical device 102. Implantable medical device 102 converts at least a portion of the received RF energy into localized treatment-level heat. In some embodiments, implantable medical device 102 transitions between a treatment-heating mode and a standby, hold, or reduced-heating mode. A treatment session lasts, for example, about twenty minutes as illustrated, less than twenty minutes, more than twenty minutes, one hour, two hours, several hours, or another clinically appropriate duration.

In some embodiments, temperature monitoring continues after treatment-level heating or administration of another therapy has ended. Temperature information is obtained periodically, intermittently, or continuously for several hours, up to about one day, or for a longer post-treatment monitoring period. The post-treatment temperature information is optionally communicated to a remote monitoring system through a wired or wireless network in some embodiments. Control system 108 compares post-treatment temperature information with a pretreatment baseline, temperature information obtained during treatment, or earlier post-treatment measurements to identify a change in local temperature or a temperature trend at or near implantable medical device 102. A post-treatment temperature change or trend is used, alone or with other clinical information, to assess a local biological response associated with inflammation, perfusion, metabolic activity, tumor response, tissue injury, or recovery. The monitored response may indicate an expected therapeutic response, an insufficient therapeutic response, or a need to modify a subsequent treatment session. In some embodiments, a lower-power RF field supplies operating power for temperature sensing and communication during post-treatment monitoring without generating treatment-level heat.

Localized heating produced by implantable medical device 102 weakens or preconditions diseased cells, modifies a tumor microenvironment, increases sensitivity of diseased cells to chemotherapy, increases perfusion or delivery of a therapeutic agent to target tissue, activates a temperature-sensitive therapeutic agent, or combinations thereof. In some embodiments, hyperthermia is applied while a patient receives chemotherapy through infusion system 906. In other embodiments, hyperthermia is applied before or after the infusion.

In one oncology scenario, implantable medical device 102 is placed in or near a tumor using image guidance, such as fluoroscopy, ultrasound, computed tomography, magnetic resonance imaging, or another imaging technique. During a subsequent outpatient treatment session, the patient is positioned in treatment chair 904, infusion system 906 administers a therapeutic agent, and radiofrequency coil 106 couples RF energy to implantable medical device 102. The localized heat increases the efficacy of chemotherapy in some embodiments while limiting heating of healthy tissue remote from implantable medical device 102.

Treatment-level heating is applied continuously or intermittently. In some embodiments, a switch initiates treatment-level heating for an interval of less than one second and then reduces or discontinues treatment-level heating while stored energy dissipates. Treatment sessions are performed once per day, once per week, or according to another schedule over several weeks or months.

In some embodiments, localized heat acts as a signal that activates a temperature-sensitive chemical reaction, temperature-sensitive drug carrier, temperature-sensitive cell, cell-based therapy, or another temperature-responsive treatment.

In some embodiments, the therapeutic agent includes doxorubicin, a temperature-sensitive formulation containing doxorubicin, or another chemotherapy agent. For example, a temperature-sensitive liposomal formulation, such as ThermoDox®, releases doxorubicin more readily at an elevated temperature than at normal body temperature. Implantable medical device 102 locally raises temperature in or near target tissue, thereby promoting localized release or activity of the therapeutic agent at the target tissue. FIG. 9 illustrates the treatment environment and temporal relationship between infusion therapy and hyperthermia, rather than the internal structure or release mechanism of the drug carrier.

Although oncology is a primary contemplated application, implantable medical device 102 is not limited to oncology. Other applications include neurological applications, quieting nerves associated with pain pathways, treating hypertension, treating inflammation, treating other conditions, or combinations thereof. For nerve-related applications, a maximum setpoint temperature is selected to avoid nerve damage. The disclosed treatments are used in human medicine or veterinary medicine to treat humans or non-human animals. Implantable medical device 102 is also used to ablate diseased cells in some embodiments.

One or more radiofrequency coils 106 are installed in or associated with treatment chair 904, a treatment bed, small-animal enclosure, wearable support, handheld applicator, or another support. Control system 108 controls an RF drive signal applied to radiofrequency coil 106, receives temperature information from implantable medical device 102, verifies treatment, and adjusts treatment parameters. In some embodiments, treatment is delivered in a clinic, infusion center, home, or another therapeutic setting without requiring MRI or ultrasound guidance during each treatment session.

Example operational parameters include external electrical drive power of about 10 W and about 10 V peak for implantable medical device 102 positioned about 3 cm below a body surface. In some embodiments, external drive power scales approximately with depth squared, such that implantable medical device 102 positioned about 9 cm below a body surface uses about 90 W of external drive power. Other power levels are used depending on implant depth, tissue characteristics, implant orientation, coupling efficiency, treatment temperature, treatment duration, and other design or treatment considerations.

In switch-controlled implementations described with reference to FIGS. 3-7, implant-side control circuitry regulates heating by controlling a switch that changes an inductance, capacitance, loading, magnetic coupling, impedance, resonant response, or energy-harvesting state of implantable medical device 102. FIGS. 10-14 emphasize a different control architecture in which temperature is sensed at implantable medical device 102, temperature information is wirelessly communicated outside the body, and control system 108 regulates heating by adjusting externally applied RF energy based on the communicated temperature information. Accordingly, implantable medical device 102 in the embodiments of FIGS. 10-14 need not include a switch-controlled heat-reduction circuit, secondary coil, choke coil, or other implant-side circuitry configured to reduce treatment-level heating.

FIG. 10 illustrates implantable medical device 102 according to a first externally regulated temperature-feedback heating embodiment 1000. Implantable medical device 102 of FIG. 10 generates localized treatment-level heat, senses temperature at or near implantable medical device 102, and wirelessly communicates temperature information to external radiofrequency applicator 104 or control system 108. Control system 108 uses the communicated temperature information as feedback to regulate RF energy applied to implantable medical device 102.

Implantable medical device 102 includes hermetically sealed enclosure 1002, magnetic core 1004, RFID ASIC 1006, implant coil arrangement 1008, and temperature sensor 1010. In the embodiment of FIG. 10, implant coil arrangement 1008 comprises a common implant coil configured both to couple RF energy used to generate treatment-level heat and to supply operating power to wireless communication circuitry 1012. RFID ASIC 1006 provides at least part of wireless communication circuitry 1012, which includes transmitter circuitry, receiver circuitry, transceiver circuitry, control circuitry, or combinations thereof configured to wirelessly communicate temperature information based on a temperature sensed by temperature sensor 1010. As described previously with reference to FIG. 3, implant coil arrangement 1008 includes a single implant coil or a plurality of functionally distinct implant coils.

Implant coil arrangement 1008 is operatively coupled to RFID ASIC 1006 and supplies electrical power harvested from an externally generated RF electromagnetic field. Wireless communication circuitry 1012 transmits and receives information using communication circuitry and an associated antenna or coupling element, which is integrated with RFID ASIC 1006, separately connected to RFID ASIC 1006, or otherwise provided within implantable medical device 102. Accordingly, operative coupling between RFID ASIC 1006 and implant coil arrangement 1008 does not require temperature information to be transmitted through implant coil arrangement 1008.

In some embodiments, the common implant coil of implant coil arrangement 1008 additionally functions as an antenna or inductive-coupling element for wireless communication circuitry 1012. For example, RFID ASIC 1006 communicates information by modulating a load, impedance, resonant response, or other electrical characteristic associated with implant coil arrangement 1008. Implant coil arrangement 1008 therefore supplies operating power to RFID ASIC 1006 and, in some embodiments, also participates in wireless communication.

RFID communication is one example implementation. In other embodiments, wireless communication circuitry 1012 includes another integrated circuit, controller, transponder, telemetry circuit, backscatter circuit, load-modulation circuit, near-field communication circuit, inductive communication circuit, or other circuitry configured to wirelessly communicate temperature information using another wireless communication protocol.

Hermetically sealed enclosure 1002 encloses magnetic core 1004, RFID ASIC 1006, implant coil arrangement 1008, temperature sensor 1010, or combinations thereof. Temperature sensor 1010 is integrated into RFID ASIC 1006, electrically coupled to RFID ASIC 1006, positioned on a common support with RFID ASIC 1006, or positioned separately within implantable medical device 102.

Temperature sensor 1010 senses a temperature at or near implantable medical device 102. The sensed temperature includes a temperature of magnetic core 1004, a temperature of implantable medical device 102, a local tissue temperature, or a value correlated with one or more of these temperatures. Wireless communication circuitry 1012 wirelessly communicates (e.g., in real time) temperature feedback based on the sensed temperature using an antenna or coupling element associated with wireless communication circuitry 1012. In some embodiments, implant coil arrangement 1008 additionally serves as the antenna or coupling element used for wireless communication.

Implant coil arrangement 1008 wirelessly couples RF energy from an RF electromagnetic field generated outside the body, such as an RF electromagnetic field generated using radiofrequency coil 106 of external radiofrequency applicator 104. The coupled RF energy supplies operating power to RFID ASIC 1006 and magnetically excites magnetic core 1004, causing magnetic core 1004 to generate localized treatment-level heat for treating target tissue. In some embodiments, implant coil arrangement 1008 additionally participates in transmission or reception of wireless communication signals.

Control system 108 receives temperature feedback communicated by wireless communication circuitry 1012 and adjusts a power level of an RF signal applied to radiofrequency coil 106 based on the temperature feedback. Adjustment of the externally applied power level regulates localized treatment-level heat generated by magnetic core 1004 and maintains a desired device temperature, setpoint temperature, treatment temperature range, thermal dose, or other treatment condition.

In the illustrated implementation, external radiofrequency applicator 104 applies a lower-power RF field at about 135 kHz during a temperature-readout operation and a higher-power RF field at about 135 kHz during a heating operation. The lower-power RF field provides sufficient energy through implant coil arrangement 1008 to power or interrogate RFID ASIC 1006 and support communication of temperature information while producing little or no treatment-level heating. The higher-power RF field produces greater magnetic excitation and magnetic loss in magnetic core 1004, thereby causing magnetic core 1004 to generate treatment-level heat.

Wireless communication circuitry 1012 need not control an implant-side heat-reduction switch to regulate treatment-level heating. Instead, control system 108 regulates heating by changing the power level of the RF signal applied to radiofrequency coil 106 based on the communicated temperature information.

Control system 108 alternates between or otherwise coordinates wireless temperature readout and application of heating energy. For example, control system 108 obtains temperature information from implantable medical device 102, determines whether the sensed temperature is below, within, or above a desired temperature or temperature range, and increases, decreases, maintains, or temporarily discontinues the power level applied to radiofrequency coil 106. Repeated temperature readout and corresponding adjustment of externally applied RF power provide closed-loop regulation of localized treatment-level heat generated by magnetic core 1004.

Although about 135 kHz and RFID communication are provided as examples, other frequencies and wireless communication protocols are used. The temperature-readout frequency and heating frequency in FIG. 10 are the same, substantially the same, within a tolerance range, or sufficiently close that implant coil arrangement 1008 couples RF energy used both to supply operating power to RFID ASIC 1006 and to generate treatment-level heat. In some embodiments, implant coil arrangement 1008 additionally functions as an antenna or inductive-coupling element used for wireless communication.

Unless otherwise indicated, the temperature sensing, wireless communication, external closed-loop regulation, protocol alternatives, and other operating principles described with reference to FIG. 10 also apply to the embodiments of FIGS. 11-14. In each of the embodiments of FIGS. 11-14, a communication coil is operatively coupled to the respective wireless communication circuitry and is configured at least to couple RF energy used to supply operating power to the wireless communication circuitry. The wireless communication circuitry communicates temperature information using transmitter circuitry, receiver circuitry, transceiver circuitry, or combinations thereof and an associated antenna or coupling element. The antenna or coupling element is integrated with the respective RFID ASIC, separately connected to the RFID ASIC, or otherwise provided within implantable medical device 102. In some embodiments, the communication coil additionally functions as the antenna or coupling element used for wireless communication. Operative coupling between the communication coil and the wireless communication circuitry therefore does not require temperature information to be transmitted through the communication coil.

FIG. 11 illustrates implantable medical device 102 according to a second externally regulated temperature-feedback heating embodiment 1100. The embodiment of FIG. 11 differs from the embodiment of FIG. 10 principally in that separate coils and different RF frequencies are used for treatment-level heating and for supplying operating power to wireless communication circuitry.

Implantable medical device 102 of FIG. 11 includes hermetically sealed enclosure 1102, magnetic core 1104, capacitor 1106, heating coil 1108, RFID ASIC 1110, communication coil 1112, and temperature sensor 1114. RFID ASIC 1110 provides at least part of wireless communication circuitry 1116. Communication coil 1112 is operatively coupled to RFID ASIC 1110 and is configured to couple RF energy that supplies operating power to RFID ASIC 1110. In some embodiments, communication coil 1112 additionally functions as an antenna or inductive-coupling element used by wireless communication circuitry 1116 to transmit or receive information.

Heating coil 1108 and capacitor 1106 form at least part of a resonant heating circuit associated with magnetic core 1104. In this embodiment, the heating element comprises magnetic core 1104 and, in some implementations, additionally comprises heating coil 1108 or another electrically resistive conductor that generates a portion of the localized treatment-level heat. Communication coil 1112 couples RF energy at a communication or temperature-readout frequency different from the heating frequency and supplies operating power to RFID ASIC 1110. Wireless communication circuitry 1116 communicates temperature information using an associated antenna or coupling element, which is separate from communication coil 1112 in some embodiments and is provided by communication coil 1112 in other embodiments.

In one implementation, external radiofrequency applicator 104 applies a lower-power RF field at about 135 kHz to supply operating power to RFID ASIC 1110 and support a temperature-readout operation, and applies a higher-power RF field at a different frequency for heating. Heating coil 1108 and capacitor 1106 are selected or tuned for the heating frequency, while communication coil 1112 is selected or tuned for the communication or temperature-readout frequency. The separate coils, different frequencies, or both at least partially isolate wireless communication circuitry 1116 from the heating element and reduce undesired coupling of heating energy into RFID ASIC 1110.

FIG. 12 illustrates implantable medical device 102 according to a third externally regulated temperature-feedback heating embodiment 1200. The embodiment of FIG. 12 is similar to the embodiment of FIG. 11, except that the heating and wireless temperature-communication functions are associated with respective magnetic cores separated by gap 1202.

Implantable medical device 102 of FIG. 12 includes gap 1202, first magnetic core 1204, capacitor 1206, heating coil 1208, second magnetic core 1210, RFID ASIC 1212, communication coil 1214, and temperature sensor 1216. RFID ASIC 1212 provides at least part of wireless communication circuitry 1218. Communication coil 1214 is operatively coupled to RFID ASIC 1212 and is configured to couple RF energy that supplies operating power to RFID ASIC 1212. In some embodiments, communication coil 1214 additionally functions as an antenna or inductive-coupling element used by wireless communication circuitry 1218.

Heating coil 1208 and capacitor 1206 form a resonant heating circuit associated with first magnetic core 1204. In this embodiment, the heating element comprises first magnetic core 1204 and, in some implementations, additionally comprises heating coil 1208 or another electrically resistive conductor that generates a portion of the localized treatment-level heat. Communication coil 1214 supplies operating power to RFID ASIC 1212 and is associated with second magnetic core 1210. Wireless communication circuitry 1218 communicates temperature information using an associated antenna or coupling element, which is separate from communication coil 1214 in some embodiments and is provided by communication coil 1214 in other embodiments. Temperature sensor 1216 is integrated into or coupled to RFID ASIC 1212, positioned near first magnetic core 1204 or heating coil 1208, positioned near second magnetic core 1210 or RFID ASIC 1212, or positioned elsewhere within implantable medical device 102. When temperature sensor 1216 is spaced from the heating element, the sensed temperature is correlated with a temperature of first magnetic core 1204, heating coil 1208, target tissue, or another treatment temperature.

Gap 1202 separates the heating portion from the wireless temperature-communication arrangement and provides physical, magnetic, electrical, electromagnetic, thermal, or combinations of isolation. Gap 1202 is an open space, a material-filled space, a discontinuity between first magnetic core 1204 and second magnetic core 1210, or another separating structure. Gap 1202 includes air, glass, ceramic, polymer, epoxy, a non-magnetic material, an electrically insulating material, a low-thermal-conductivity material, or combinations thereof. The separate cores and gap 1202 reduce magnetic coupling between heating coil 1208 and communication coil 1214 and reduce undesired coupling of heating energy into RFID ASIC 1212.

FIG. 13 illustrates implantable medical device 102 according to a fourth externally regulated temperature-feedback heating embodiment 1300. The embodiment of FIG. 13 is similar to the embodiment of FIG. 12, except that the heating portion and wireless temperature-communication portion are physically spaced apart without requiring a separately identified gap.

Implantable medical device 102 of FIG. 13 includes first magnetic core 1302, capacitor 1304, heating coil 1306, second magnetic core 1308, RFID ASIC 1310, communication coil 1312, and temperature sensor 1314. RFID ASIC 1310 provides at least part of wireless communication circuitry 1316. Communication coil 1312 is operatively coupled to RFID ASIC 1310 and is configured to couple RF energy that supplies operating power to RFID ASIC 1310. In some embodiments, communication coil 1312 additionally functions as an antenna or inductive-coupling element used by wireless communication circuitry 1316.

First magnetic core 1302, capacitor 1304, and heating coil 1306 form a heating portion positioned in a first side, end, portion, or region of implantable medical device 102. Second magnetic core 1308, RFID ASIC 1310, communication coil 1312, and temperature sensor 1314 form at least part of a temperature-monitoring portion positioned in a second side, end, portion, or region of implantable medical device 102. Communication coil 1312 supplies operating power to RFID ASIC 1310. Wireless communication circuitry 1316 communicates temperature information using an associated antenna or coupling element, which is separate from communication coil 1312 in some embodiments and is provided by communication coil 1312 in other embodiments.

The heating portion and wireless temperature-communication portion are separated by a selected distance. The intervening space includes an unoccupied region, enclosure material, air, glass, ceramic, polymer, epoxy, a non-magnetic material, an electrically insulating material, a low-thermal-conductivity material, or combinations thereof. The spacing reduces undesired magnetic, electrical, electromagnetic, or thermal coupling between the respective portions and reduces undesired coupling of heating energy into RFID ASIC 1310.

The spacing and resulting geometry are selected based on implantation approach, tissue target, available anatomical space, heating efficiency, temperature-measurement accuracy, RF coupling, communication reliability, isolation, implant retention, mechanical stability, or combinations thereof. The spaced arrangement provides, in some embodiments, an implantable medical device 102 having a different length, width, aspect ratio, or overall geometry than the embodiments of FIGS. 10-12 and 14.

For each of the embodiments of FIGS. 11-14, the respective communication coil couples RF energy used to supply operating power to the respective wireless communication circuitry, and the respective heating coil couples RF energy used to generate localized treatment-level heat. Control system 108 receives temperature information communicated by the respective wireless communication circuitry and adjusts RF power coupled through the respective heating coil. In some embodiments, the communication coil also functions as an antenna or inductive-coupling element used to transmit or receive the temperature information. In other embodiments, the wireless communication circuitry communicates through a separate antenna or coupling element. Control system 108 thereby regulates localized treatment-level heat as described with reference to FIG. 10. The heating and communication frequencies are selected based on heating efficiency, communication reliability, desired frequency separation, magnetic isolation, tissue absorption, implant dimensions, component ratings, safety limits, regulatory requirements, or combinations thereof. The heating frequency is higher than, lower than, or otherwise different from the communication frequency. Other wireless communication protocols and frequency arrangements are used in other embodiments.

The externally regulated arrangements of FIGS. 10-14 are also used in treatment systems that include implant-side regulation. For example, in the multiple-implant arrangement of FIG. 2, temperature information communicated by one or more implantable medical devices 102 is used by control system 108 to adjust RF power applied by external radiofrequency applicator 104, while one or more implantable medical devices 102 independently reduce or discontinue treatment-level heating when a locally sensed temperature reaches or exceeds a threshold. External closed-loop regulation thereby controls RF energy applied to implantable medical devices 102 collectively or regionally, while implant-side regulation provides local temperature control or over-temperature protection for individual implantable medical devices 102.

The externally regulated implant architectures described with reference to FIGS. 10-14 are also used in the multiple-implant treatment system of FIG. 2. In some such embodiments, respective implantable medical devices 102 are tuned to different heating frequencies, while their wireless communication circuitry operates at a common communication or temperature-readout frequency. Control system 108 selectively obtains temperature information from each implantable medical device 102 and independently adjusts RF energy applied at the heating frequency associated with that implantable medical device 102. RF energy at the respective heating frequencies is applied sequentially, simultaneously, or according to another coordinated sequence.

FIG. 14 illustrates implantable medical device 102 according to a fifth externally regulated temperature-feedback heating embodiment 1400. Implantable medical device 102 includes hermetically sealed enclosure 1402, magnetic core 1404, capacitor 1406, heating coil 1408, resistor 1410, RFID ASIC 1412, temperature sensor 1414, communication coil 1416, and wireless communication circuitry 1418.

Heating coil 1408 and capacitor 1406 form at least part of a resonant heating circuit configured to couple RF energy at a heating frequency. Heating coil 1408 has a non-magnetic interior region and is not wound around a magnetic heating core. Alternating electrical current induced in heating coil 1408 generates heat through electrical resistance of the conductor forming heating coil 1408. In the illustrated embodiment, resistive losses in heating coil 1408 generate most of the localized treatment-level heat.

The resistance, conductor material, conductor length, conductor cross-sectional area, number of turns, winding geometry, operating frequency, resonant response, Q factor, and other characteristics of heating coil 1408 are selected to provide a desired rate and amount of localized heating. Heating coil 1408 includes wire, a conductive trace, a deposited conductor, a printed conductor, a thin-film conductor, or another electrically conductive winding having an electrical resistance selected to generate treatment-level heat.

Resistor 1410 is electrically coupled to heating coil 1408 and contributes additional resistive heating in some embodiments. Resistor 1410 includes a discrete resistor, resistive conductor, resistive trace, thin-film resistor, conductive polymer, or another electrically resistive component. In some embodiments, heating coil 1408 generates a majority of the localized treatment-level heat and resistor 1410 generates a remaining portion, collectively forming a heating element 1420. In other embodiments, resistor 1410 generates a majority of the localized treatment-level heat for heating element 1420. Resistor 1410 is omitted in some implementations in which the resistance of heating coil 1408 provides the desired heating element 1420.

Communication coil 1416 is associated with magnetic core 1404 and is operatively coupled to RFID ASIC 1412. Communication coil 1416 couples RF energy at a communication or temperature-readout frequency and supplies operating power to RFID ASIC 1412 and wireless communication circuitry 1418. Magnetic core 1404 improves magnetic coupling, energy collection, voltage generation, or operating-power efficiency of communication coil 1416 in some embodiments. Magnetic core 1404 is associated primarily with the communication and temperature-readout functions and need not provide a substantial portion of the localized treatment-level heat.

Temperature sensor 1414 senses a temperature of heating coil 1408, resistor 1410, implantable medical device 102, local tissue, or a value correlated with one or more of these temperatures. Temperature sensor 1414 is integrated into RFID ASIC 1412, electrically coupled to RFID ASIC 1412, positioned adjacent heating coil 1408 or resistor 1410, or positioned elsewhere within enclosure 1402. Wireless communication circuitry 1418 communicates temperature feedback based on the sensed temperature to control system 108.

In one implementation, external radiofrequency applicator 104 applies a lower-power RF field at about 135 kHz to communication coil 1416 during a temperature-readout operation. The lower-power RF field supplies operating power to RFID ASIC 1412 and supports communication of temperature information. External radiofrequency applicator 104 applies a higher-power RF field at a heating frequency different from the communication frequency to heating coil 1408 during a heating operation. The heating frequency is selected to cause heating coil 1408 and, when present, resistor 1410 to generate localized treatment-level heat. About 135 kHz is an example communication frequency, and other communication and heating frequencies are used in other embodiments.

The use of separate coils and different frequencies at least partially isolates RFID ASIC 1412 and wireless communication circuitry 1418 from RF energy used for treatment-level heating. The frequency separation, physical spacing, separate resonant circuits, magnetic core 1404 associated with communication coil 1416, non-magnetic interior region of heating coil 1408, or combinations thereof reduce undesired coupling of heating-frequency energy into RFID ASIC 1412 and reduce or prevent overheating, electrical overstress, or damage to RFID ASIC 1412.

Control system 108 obtains temperature feedback during a temperature-readout operation and adjusts a power level, duty cycle, application duration, pulse interval, or another operating parameter of RF energy applied at the heating frequency. Regulation of heating is therefore performed through adjustment of externally generated RF energy rather than through operation of an implant-side heat-reduction switch. The temperature-readout operation and the heating operation are repeated during treatment according to a coordinated sequence, which may include sequential, simultaneous, or intermittent operation.

The embodiment of FIG. 14 is also used in the multiple-implant arrangement of FIG. 2. Respective implantable medical devices 102 receive operating power and communicate temperature information at a common communication frequency, while different implantable medical devices 102 receive treatment-level heating energy at different heating frequencies. Control system 108 selectively obtains temperature information from each implantable medical device 102 and independently adjusts RF energy applied at the corresponding heating frequency.

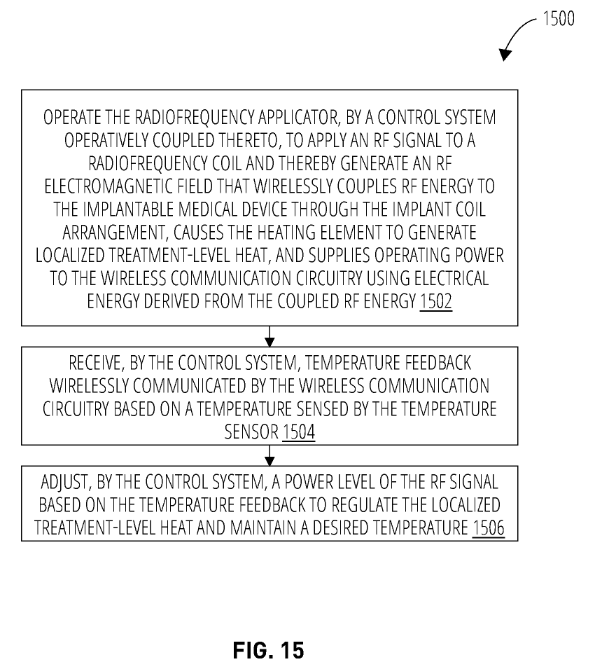

FIG. 15 illustrates process 1500 for controlling hyperthermia treatment of target tissue in a body. Process 1500 is performed using a treatment system comprising implantable medical device 102 positioned in or adjacent to the target tissue, external radiofrequency applicator 104 positioned outside the body, and control system 108 operatively coupled to external radiofrequency applicator 104. Implantable medical device 102 comprises a heating element, an implant coil arrangement, a temperature sensor, and wireless communication circuitry. The heating element comprises a magnetically lossy material, an electrically resistive conductor, or both.

Implantable medical device 102 is positioned in or adjacent to the target tissue before or in connection with a treatment session. External radiofrequency applicator 104 is positioned outside the body so that radiofrequency coil 106 wirelessly couples RF energy to implantable medical device 102. These positioning operations establish the treatment arrangement in which process 1500 is performed and are not required to be performed by control system 108 as part of process 1500.

At block 1502, control system 108 operates external radiofrequency applicator 104 to apply an RF signal to radiofrequency coil 106 and thereby generate an RF electromagnetic field. The RF electromagnetic field wirelessly couples RF energy to implantable medical device 102 through the implant coil arrangement, causes the heating element to generate localized treatment-level heat, and supplies operating power to the wireless communication circuitry using electrical energy derived from the coupled RF energy.

The heating element generates localized treatment-level heat through magnetic losses, resistive losses, or a combination of magnetic and resistive losses. In some embodiments, the heating element comprises a magnetically lossy core, such as a ferrite core. In other embodiments, the heating element comprises a heating coil, resistor, resistive conductor, resistive trace, or another electrically resistive component. The heating element is separate from the implant coil arrangement in some embodiments and comprises at least a portion of the implant coil arrangement in other embodiments.

At block 1504, control system 108 receives temperature feedback wirelessly communicated by the wireless communication circuitry based on a temperature sensed by the temperature sensor. The sensed temperature comprises a temperature of the heating element, a temperature of implantable medical device 102, a local tissue temperature, or a value correlated with one or more of these temperatures.

At block 1506, control system 108 adjusts a power level of the RF signal based on the temperature feedback to regulate the localized treatment-level heat and maintain a desired temperature. The desired temperature comprises a setpoint temperature, treatment temperature range, maximum temperature, thermal-dose condition, or another treatment condition. Control system 108 increases, decreases, maintains, or temporarily discontinues the RF power based on the temperature feedback.

Blocks 1502–1506 are repeated during a treatment session to provide closed-loop temperature regulation. Control system 108 receives successive temperature-feedback values and repeatedly adjusts the RF signal based on the successive temperature-feedback values. The illustrated order is an example. Temperature feedback is received while RF energy is being applied, during an interval between applications of treatment-level RF energy, or according to another coordinated sequence.

In some embodiments, control system 108 operates external radiofrequency applicator 104 to repeatedly perform a temperature-readout operation and a heating operation at the same or substantially the same frequency. During the temperature-readout operation, external radiofrequency applicator 104 applies a lower-power RF field sufficient to supply operating power to the wireless communication circuitry and obtain the temperature feedback while producing little or no localized treatment-level heat. During the heating operation, external radiofrequency applicator 104 applies a higher-power RF field sufficient to cause the heating element to generate localized treatment-level heat. The temperature-readout operation and the heating operation are performed in either order and need not follow a fixed sequence.

In other embodiments, the implant coil arrangement comprises a communication coil and a heating coil. Control system 108 operates external radiofrequency applicator 104 to apply RF energy at a communication frequency. The RF energy at the communication frequency is coupled through the communication coil and supplies operating power to the wireless communication circuitry. Control system 108 also operates external radiofrequency applicator 104 to apply RF energy at a heating frequency different from the communication frequency. The RF energy at the heating frequency is coupled through the heating coil and causes the heating element to generate localized treatment-level heat. RF energy at the communication frequency and RF energy at the heating frequency are applied simultaneously or at different times.

In some separate-coil embodiments, the heating element comprises a magnetic core associated with the heating coil. In other separate-coil embodiments, the heating element comprises an electrically resistive conductor forming at least a portion of the heating coil, and resistive losses in the heating coil generate a majority of the localized treatment-level heat. A resistor or another electrically resistive component coupled to the heating coil generates an additional portion of the localized treatment-level heat in some embodiments. The communication coil is associated with a magnetic core in some implementations.

For example, in the embodiment described with reference to FIG. 14, the heating coil has a non-magnetic interior region and generates localized treatment-level heat primarily through resistive losses. A separate communication coil is associated with a magnetic core and supplies operating power to the wireless communication circuitry at a communication frequency different from the heating frequency.

In some embodiments, implantable medical device 102 is a first implantable medical device configured to receive treatment-level heating energy at a first heating frequency, and the treatment system further comprises a second implantable medical device configured to receive treatment-level heating energy at a second heating frequency different from the first heating frequency. Control system 108 operates external radiofrequency applicator 104 to apply RF energy at the first and second heating frequencies, receives respective temperature feedback from the first and second implantable medical devices, and independently adjusts RF power applied at the first and second heating frequencies based on the respective temperature feedback.

The first and second implantable medical devices receive operating power for temperature sensing and wireless communication at a common temperature-readout frequency in some embodiments. To reduce or prevent overlapping communications, control system 108 selectively obtains temperature feedback from a selected implantable medical device.

In one implementation, control system 108 operates external radiofrequency applicator 104 to transmit an implant-specific identifier, address, command, or other downlink signal at the common temperature-readout frequency. The selected implantable medical device communicates its temperature feedback while another implantable medical device remains silent.

In another implementation, control system 108 operates external radiofrequency applicator 104 to apply RF energy at the common temperature-readout frequency together with reduced-power RF energy at the heating frequency associated with the selected implantable medical device. The associated heating frequency identifies or enables the selected implantable medical device while producing little or no localized treatment-level heat. The implant-specific command technique and the frequency-selective technique are used separately or together.

In some embodiments, the wireless communication circuitry comprises an RFID ASIC. The RFID ASIC comprises transmitter circuitry, receiver circuitry, transceiver circuitry, control circuitry, or combinations thereof. Operation of external radiofrequency applicator 104 supplies operating power to the RFID ASIC using electrical energy derived from RF energy coupled through the implant coil arrangement or communication coil. The RFID ASIC communicates temperature feedback using load modulation, backscatter communication, RFID communication, or another wireless communication technique.

Process 1500 continues until a desired temperature, treatment duration, cumulative thermal dose, or other treatment endpoint is reached. Control system 108 additionally adjusts a frequency, duty cycle, application duration, readout interval, heating interval, position, orientation, or another treatment parameter based on the temperature feedback or other communicated information in some embodiments.

Control functions of process 1500 are implemented using stored instructions, firmware, hardwired logic, programmable logic, ASICs, FPGAs, processors, or combinations thereof. Features, operating modes, and process steps described in connection with separate embodiments of process 1500 are combined unless their combination is incompatible with the disclosed operation.

Skilled persons considering this disclosure will appreciate that many changes to the details of the above-described embodiments are possible without departing from the underlying principles of the invention. The scope of the claimed invention should, therefore, be determined only by the following claims and equivalents.

What is claimed is:

1. A hyperthermia treatment system comprising:
an implantable medical device configured to be positioned in or adjacent to target tissue in a body, the implantable medical device comprising:
a heating element configured to convert energy derived from coupled RF energy into localized treatment-level heat for treating the target tissue;
an implant coil arrangement configured to wirelessly couple RF energy from an RF electromagnetic field generated outside the body to the implantable medical device;
a temperature sensor configured to sense a temperature at or near the implantable medical device; and
wireless communication circuitry configured to wirelessly communicate temperature feedback based on the sensed temperature, wherein the wireless communication circuitry is operatively coupled to the implant coil arrangement to receive operating power derived from the coupled RF energy;
a radiofrequency applicator configured to be positioned outside the body, the radiofrequency applicator comprising a radiofrequency coil configured to generate the RF electromagnetic field and wirelessly couple RF energy to the implantable medical device; and
a control system operatively coupled to the radiofrequency applicator and configured to receive the temperature feedback from the implantable medical device and adjust a power level of an RF signal applied to the radiofrequency coil based on the temperature feedback to regulate the localized treatment-level heat generated by the heating element and maintain a desired temperature.

2. The hyperthermia treatment system of claim 1, wherein the heating element comprises a core including a magnetically lossy material.

3. The hyperthermia treatment system of claim 1, wherein the heating element comprises an electrically resistive conductor.

4. The hyperthermia treatment system of claim 1, wherein the heating element comprises a magnetic core and an electrically resistive conductor that each generate a portion of the localized treatment-level heat.

5. The hyperthermia treatment system of claim 1, wherein the implantable medical device is a single implantable medical device, wherein the temperature feedback corresponds to a temperature of a single heating element of the single implantable medical device, and wherein the control system adjusts the power level based on the temperature of the single heating element.

6. The hyperthermia treatment system of claim 1, wherein the wireless communication circuitry comprises an RFID ASIC configured to receive operating power derived from RF energy coupled through the implant coil arrangement.

7. The hyperthermia treatment system of claim 1, wherein the implant coil arrangement comprises a common implant coil configured both to couple RF energy used to generate the localized treatment-level heat and to supply operating power to the wireless communication circuitry.

8. The hyperthermia treatment system of claim 7, wherein the common implant coil additionally functions as an antenna or inductive-coupling element through which the wireless communication circuitry communicates the temperature feedback.

9. The hyperthermia treatment system of claim 1, wherein the implant coil arrangement comprises:
a communication coil configured to couple RF energy at a communication frequency to supply operating power to the wireless communication circuitry; and
a heating coil configured to couple RF energy at a heating frequency different from the communication frequency for generating the localized treatment-level heat.

10. The hyperthermia treatment system of claim 9, wherein the heating element comprises a first magnetic core associated with the heating coil, and wherein the implantable medical device further comprises a second magnetic core associated with the communication coil, the first magnetic core and the second magnetic core being separated by a gap or selected spacing.

11. The hyperthermia treatment system of claim 9, wherein the heating element comprises an electrically resistive conductor forming at least a portion of the heating coil, wherein resistive losses in the heating coil generate a majority of the localized treatment-level heat, and wherein the communication coil is associated with a magnetic core.

12. The hyperthermia treatment system of claim 1, wherein the implantable medical device is a first implantable medical device configured to receive treatment-level heating energy at a first heating frequency, the system further comprising a second implantable medical device comprising:
a second heating element;
a second implant coil arrangement configured to couple RF energy at a second heating frequency different from the first heating frequency, wherein the second heating element is configured to generate localized treatment-level heat in response to the RF energy coupled through the second implant coil arrangement;
a second temperature sensor configured to sense a temperature at or near the second implantable medical device; and
second wireless communication circuitry configured to wirelessly communicate second temperature feedback based on the temperature sensed by the second temperature sensor,
wherein the control system is configured to receive respective temperature feedback from the first and second implantable medical devices and independently adjust RF power applied at the first and second heating frequencies based on the respective temperature feedback.

13. The hyperthermia treatment system of claim 12, wherein the wireless communication circuitry of the first implantable medical device and the second wireless communication circuitry are configured to receive operating power at a common temperature-readout frequency, and wherein the control system is configured to selectively obtain temperature feedback from the first and second implantable medical devices.

14. The hyperthermia treatment system of claim 13, wherein the control system is configured to selectively obtain temperature feedback from a selected one of the first and second implantable medical devices by causing the radiofrequency applicator to transmit an implant-specific command at the common temperature-readout frequency.

15. The hyperthermia treatment system of claim 13, wherein the control system is configured to selectively obtain temperature feedback from a selected one of the first and second implantable medical devices by causing the radiofrequency applicator to apply RF energy at the common temperature-readout frequency together with reduced-power RF energy at the heating frequency associated with the selected one of the first and second implantable medical devices.

16. An implantable medical device for use in a hyperthermia treatment system, the implantable medical device comprising:
an enclosure configured for implantation in a body;
a heating element positioned within the enclosure;
an implant coil arrangement positioned within the enclosure and configured to wirelessly couple RF energy from an RF electromagnetic field generated outside the body to the implantable medical device, wherein the heating element is configured to convert energy derived from the coupled RF energy into localized treatment-level heat;
a temperature sensor configured to sense a temperature at or near the implantable medical device; and
wireless communication circuitry positioned within the enclosure and configured to wirelessly communicate temperature feedback based on the sensed temperature for use by a control system in adjusting RF energy applied to the implantable medical device, wherein the wireless communication circuitry is operatively coupled to the implant coil arrangement to receive operating power derived from the coupled RF energy.

17. The implantable medical device of claim 16, wherein the wireless communication circuitry comprises an RFID ASIC including transmitter circuitry, receiver circuitry, transceiver circuitry, or combinations thereof.

18. The implantable medical device of claim 16, wherein the implant coil arrangement comprises a common implant coil configured both to couple RF energy used to generate the localized treatment-level heat and to supply operating power to the wireless communication circuitry.

19. The implantable medical device of claim 18, wherein the common implant coil additionally functions as an antenna or inductive-coupling element through which the wireless communication circuitry communicates the temperature feedback.

20. The implantable medical device of claim 16, wherein the implant coil arrangement comprises:
a communication coil configured to couple RF energy at a communication frequency to supply operating power to the wireless communication circuitry; and
a heating coil configured to couple RF energy at a heating frequency different from the communication frequency for generating the localized treatment-level heat.

21. The implantable medical device of claim 20, wherein the heating element comprises a first magnetic core associated with the heating coil, and wherein the implantable medical device further comprises a second magnetic core associated with the communication coil, the first magnetic core and the second magnetic core being separated by a gap or selected spacing.

22. The implantable medical device of claim 20, wherein the heating element comprises an electrically resistive conductor forming at least a portion of the heating coil, wherein resistive losses in the heating coil generate a majority of the localized treatment-level heat, and wherein the communication coil is associated with a magnetic core.

23. The implantable medical device of claim 18, wherein the common implant coil is configured to receive RF energy at a frequency during a temperature-readout operation and during a heating operation, wherein the RF energy has a first power level during the temperature-readout operation sufficient to supply operating power to the wireless communication circuitry and a second, higher power level during the heating operation sufficient to generate the localized treatment-level heat, and wherein the wireless communication circuitry is configured to communicate the temperature feedback during the temperature-readout operation.

24. A method of controlling hyperthermia treatment of target tissue in a body using a treatment system comprising an implantable medical device positioned in or adjacent to the target tissue and a radiofrequency applicator positioned outside the body, the implantable medical device comprising a heating element, an implant coil arrangement, a temperature sensor, and wireless communication circuitry, the method comprising:
operating, by a control system operatively coupled to the radiofrequency applicator, the radiofrequency applicator to apply an RF signal to a radiofrequency coil and thereby generate an RF electromagnetic field that wirelessly couples RF energy to the implantable medical device through the implant coil arrangement, causes the heating element to generate localized treatment-level heat, and supplies operating power to the wireless communication circuitry using electrical energy derived from the coupled RF energy;
receiving, by the control system, temperature feedback wirelessly communicated by the wireless communication circuitry based on a temperature sensed by the temperature sensor; and
adjusting, by the control system, a power level of the RF signal based on the temperature feedback to regulate the localized treatment-level heat and maintain a desired temperature.

25. The method of claim 24, further comprising operating, by the control system, the radiofrequency applicator to repeatedly perform a temperature-readout operation and a heating operation at a frequency, wherein the temperature-readout operation comprises applying a lower-power RF field sufficient to supply operating power to the wireless communication circuitry and obtain the temperature feedback, and wherein the heating operation comprises applying a higher-power RF field sufficient to generate the localized treatment-level heat.

26. The method of claim 24, wherein the implant coil arrangement comprises a communication coil and a heating coil, the method further comprising:
operating, by the control system, the radiofrequency applicator to apply RF energy at a communication frequency, the RF energy at the communication frequency being coupled through the communication coil to supply operating power to the wireless communication circuitry; and
operating, by the control system, the radiofrequency applicator to apply RF energy at a heating frequency different from the communication frequency, the RF energy at the heating frequency being coupled through the heating coil to generate the localized treatment-level heat.

27. The method of claim 26, wherein the heating element comprises an electrically resistive conductor forming at least a portion of the heating coil, wherein resistive losses in the heating coil generate a majority of the localized treatment-level heat, and wherein the communication coil is associated with a magnetic core.

28. The method of claim 24, wherein the implantable medical device is a first implantable medical device configured to receive treatment-level heating energy at a first heating frequency, and wherein the treatment system further comprises a second implantable medical device comprising a second heating element, a second implant coil arrangement configured to couple RF energy at a second heating frequency different from the first heating frequency, a second temperature sensor, and second wireless communication circuitry, the second heating element being configured to generate localized treatment-level heat in response to the RF energy coupled through the second implant coil arrangement and the second wireless communication circuitry being configured to wirelessly communicate second temperature feedback based on a temperature sensed by the second temperature sensor, the method further comprising:
operating, by the control system, the radiofrequency applicator to apply RF energy at the first heating frequency and the second heating frequency;
receiving, by the control system, first temperature feedback from the first implantable medical device and the second temperature feedback from the second implantable medical device; and
independently adjusting, by the control system, RF power applied at the first heating frequency and the second heating frequency based on the first temperature feedback and the second temperature feedback, respectively.

29. The method of claim 28, wherein the control system selectively obtains temperature feedback from a selected one of the first and second implantable medical devices by operating the radiofrequency applicator to transmit an implant-specific command at a common temperature-readout frequency, by operating the radiofrequency applicator to apply RF energy at the common temperature-readout frequency together with reduced-power RF energy at the heating frequency associated with the selected implantable medical device, or by performing both operations.

30. The method of claim 24, wherein receiving the temperature feedback comprises receiving the temperature feedback from an RFID ASIC comprising transmitter circuitry, receiver circuitry, transceiver circuitry, or combinations thereof, and wherein the RFID ASIC receives operating power derived from RF energy coupled through the implant coil arrangement.
